(19) United States
(12) Patent Application Publication
(10) Pub. No.: US 2026/0115180 A1
(43) Pub. Date: Apr. 30, 2026

(54) TREATMENT OF HYPOTHALAMIC OBESITY

(71) Applicant: Saniona A/S, Glostrup (DK)

(72) Inventors: Jørgen DREJER, Glostrup (DK); Kim KROGSGAARD, Glostrup (DK); Berit EDSBERG, Glostrup (DK)

(21) Appl. No.: 17/996,272

(22) PCT Filed: Apr. 22, 2021
(86) PCT No.: PCT/EP2021/060548

§ 371 Date: Oct. 14, 2022

(30) Foreign Application Priority Data
Apr. 22, 2020 (EP) 20170952.4
Nov. 23, 2020 (EP) 20209186.4

(51) Int. Cl. A61K 31/46; A61P 3/04

(52) U.S. Cl. CPC A61K 31/46; A61P 3/04

(57) ABSTRACT

The present disclosure relates to treatment of Hypothalamic Obesity with Tesofensine alone or in combination with a beta-blocker. In particular the disclosure relates to treatments that lead to weight loss, loss of fat mass, in particular visceral fat, and to reduction of symptoms of pre-diabetes in patients suffering from Hypothalamic Obesity.

### CROSS-REFERENCE TO RELATED APPLICATIONS

This application is the US National Stage of International Patent Application No. PCT/EP2021/060548, filed Apr. 22, 2021, which claims priority to European Patent Application No. 20170952.4, filed Apr. 22, 2020; and European Patent Application No. 20209186.4, filed Nov. 23, 2020, the disclosures of which are incorporated herein by reference in their entireties for any and all purposes.

### TECHNICAL FIELD

The present disclosure relates to treatment of Hypothalamic Obesity, in particular to treatments that lead to weight loss, loss of fat mass, in particular visceral fat, and to reduction of symptoms of pre-diabetes in patients suffering from Hypothalamic Obesity.

### BACKGROUND

Hypothalamic Obesity (HO) is a rare disease characterized by a constant craving for food with severe consequences for patients. Hypothalamic Obesity can be the result of damage to the hypothalamus e.g. from the growth or surgical removal of a rare brain tumor, and from other types of injury to the hypothalamus including stroke, brain trauma or radiation for cancer patients. The hypothalamus is a small nucleus in the brain that controls important biological functions including body temperature, hunger and body weight. A rare brain tumor, craniopharyngioma, or the surgical removal of the tumor, is the most common cause of Hypothalamic Obesity. Hypothalamic Obesity is therefore sometimes also referred to as craniopharyngioma associated obesity.

A craniopharyngioma is a benign tumor, which most commonly affects children between 5-10 years of age, though onset can sometimes occur during adulthood. Craniopharyngioma is also a rare disease with an estimated prevalence of 1:50,000 in the US. The treatment involves surgical removal of the tumor in almost all patients. The procedure can lead to complications, including damage to the hypothalamus resulting in loss of appetite control, insatiable hunger and morbid obesity. A high frequency of Hypothalamic Obesity, between 30% and 77%, has been reported following treatment. Due to the Prader-Willi Syndrome-like insatiable hunger, Hypothalamic Obesity is sometimes referred to as “acquired Prader-Willi Syndrome”.

To date, no viable long-term solution for HO has been found, due either to the requirement of intact hypothalamic pathways or to significant side effects (Abuzzahab et al, Hypothalamic Obesity: Prologue and Promise, Horm Res Paediatr. 2019; 91 (2): 128-136. doi: 10.1159/000496564. Epub 2019 Mar. 18).

Tesofensine, i.e. [(1R,2R,3S,5S)-3-(3,4-dichlorophenyl)-2-(ethoxymethyl)-8-methyl-8-azabicyclo[3.2.1]octane], first described in WO 97/30997, is a triple monoamine reuptake inhibitor in development for the treatment of obesity.

Tesofensine effectively produces a weight loss in obese individuals of about twice of that seen with currently marketed anti-obesity drugs. Results from clinical studies with Tesofensine also showed that the compound has a good safety profile and is well tolerated. However, although no clinically relevant cardiovascular adverse events were seen, an increase in heart rate and at higher doses also small increases in blood pressure were observed. Although such small effects have no immediate risk to the patient, some medical and regulatory concerns have been raised based on observational studies, that even small changes in cardiovascular parameters may have long term implications on patients' benefit/risk evaluation.

Preclinical and clinical data suggest that appetite suppression is an important mechanism by which Tesofensine exerts its robust weight reducing effect. Notably, the strong hypophagic response (i.e. less appetite, decreased feeding, decreased craving for sweet and sugar) to Tesofensine treatment is demonstrated to be linked to central stimulation of serotonergic, noradrenergic and dopaminergic neurotransmission. However, the sympathomimetic mode of action of Tesofensine may also associate with the elevated heart rate and blood pressure observed in clinical settings.

Beta blockers, (β-blockers, beta-adrenergic blocking agents, beta antagonists, beta-adrenergic antagonists, beta-adrenoreceptor antagonists, or beta adrenergic receptor antagonists) are a class of drugs that are typically used for the management of cardiac arrhythmias, protecting the heart from a second heart attack (myocardial infarction) after a first heart attack (secondary prevention), and, in certain cases, hypertension. Beta blockers are also well known for their reductive effect on heart rate.

Metoprolol, i.e. 1-(Isopropylamino)-3-[4-(2-methoxyethyl)-phenoxy]-propan-2-ol, branded under various trade names, is a selective β1 (adrenergic) receptor blocker normally used in the treatment of various disorders of the cardiovascular system, and in particular hypertension.

Carvedilol ((±)-[3-(9H-carbazol-4-yloxy)-2-hydroxypropyl][2-(2-methoxyphenoxy)ethyl]amine) is a mixed, i.e. nonselective alpha and beta blocker. It is marketed under various trade names and is traditionally used in the treatment of mild to severe congestive heart failure (CHF) and high blood pressure.

WO 2013/120935 describes treatment of obesity by co-administration of tesofensine and metoprolol in order to ameliorate drug-induced elevation of blood pressure or increase in heart rate.

The serum half-life of tesofensine is nine days (Bara-Jimenez W, Dimitrova T, Sherzai A, Favit A, Mouradian M M, Chase T N (2004). “Effect of monoamine reuptake inhibitor NS 2330 in advanced Parkinson's disease”. Mov Disord 19 (10): 1183-6.). In comparison, the half-life of beta blockers is quite short with metoprolol in the order of 3-4 hours and carvedilol about 7 to 10 hours. Therefore, simultaneous daily administration of these two drugs is likely to induce high fluctuations in the serum levels of the beta blocker and potentially recurrent temporary absence of therapeutic efficacy of the beta blocker.

### SUMMARY

The invention relates to a method for treatment of hypothalamic obesity comprising daily administration of a pharmaceutical composition comprising 0.01 to 1.5 mg tesofensine or a pharmaceutically acceptable salt thereof to a patient suffering from hypothalamic obesity.

The present inventors have carried out a clinical phase 2 study in Hypothalamic Obesity patients with a low dose of Tesofensine and have shown statistically significant and clinically relevant reductions in body weight, waist circumference, body fat mass, and HbA1c levels. Tesofensine was co-administered with an extended release formulation of Metoprolol to counteract the effects of Tesofensine on heart rate and blood pressure.

In this treatment-resistant patient population, the treatment was efficacious and well tolerated with very few side effects. Notably there was no clinically meaningful difference in heart rate or blood pressure between the treatment groups, proving that the amounts of Tesofensine and Metoprolol were well balanced.

In embodiments of the disclosure, the body weight of the patient is reduced by at least 3% after six months of treatment, such as between 5% and 10% or between 6% and 8%.

In other embodiments, the waist circumference of the patient is reduced by at least 4 cm after 6 months of treatment, such as between 4 and 6 cm or between 6 and 10 cm. The reduction in waist circumference reflects the loss of visceral fat.

In further embodiments, the fat mass of the patient is reduced by at least 2 kg after 6 months of treatment, such as between 2 and 8 kg, or between 3 and 6 kg. Following prolonged treatment (6-12 months) there is a tendency towards an increase in lean body mass, suggesting a build-up of muscle after the initial loss of fat mass.

The reduction in HbA1c is evidence that symptoms of pre-diabetes or diabetes can be reduced in the patients. In further embodiments, the treatment reduces one or more symptoms of pre-diabetes, diabetes, metabolic syndrome, dyslipidemia, atherosclerosis, overeating, bulimia nervosa, binge eating disorder, compulsive over-eating, impaired appetite regulation, nonalcoholic fatty liver disease (NAFLD) and nonalcoholic steatohepatitis (NASH).

In one aspect the invention relates to a method for reducing body weight in a patient suffering from Hypothalamic obesity, the method comprising daily administration of a pharmaceutical composition comprising 0.01 to 1.5 mg tesofensine or a pharmaceutically acceptable salt thereof to said patient.

In one aspect the invention relates to a method for reducing waist circumference in a patient suffering from Hypothalamic obesity, the method comprising daily administration of a pharmaceutical composition comprising 0.01 to 1.5 mg tesofensine or a pharmaceutically acceptable salt to said patient.

In one aspect the invention relates to a method for reducing body fat in a patient suffering from Hypothalamic obesity, the method comprising daily administration of a pharmaceutical composition comprising 0.01 to 1.5 mg tesofensine or a pharmaceutically acceptable salt to said patient. The body fat may be visceral fat.

In one aspect the invention relates to a method for reducing liver fat in a patient suffering from Hypothalamic obesity, the method comprising daily administration of a pharmaceutical composition comprising 0.01 to 1.5 mg tesofensine or a pharmaceutically acceptable salt to said patient.

In one aspect the invention relates to a method for reducing serum HbA1c level in a patient suffering from Hypothalamic obesity, the method comprising daily administration of a pharmaceutical composition comprising 0.01 to 1.5 mg tesofensine or a pharmaceutically acceptable salt to said patient. Preferably, this patient suffers from type 2 diabetes, pre-diabetes, metabolic syndrome, insulin resistance, or glucose intolerance, preferably type 2 diabetes.

In one aspect, the present invention relates to pharmaceutical composition comprising 0.01 to 1.5 mg tesofensine, or a pharmaceutically acceptable salt thereof, for use in the treatment of hypothalamic obesity in a subject suffering from hypothalamic obesity.

In one aspect, the invention relates to use of a pharmaceutical composition comprising 0.01 to 1.5 mg tesofensine, or a pharmaceutically acceptable salt thereof, in the manufacture of a medicament for treatment of hypothalamic obesity.

### DESCRIPTION OF DRAWINGS

FIG. 1: Comparison of weight loss in Tesomet (combination of Tesofensine and Metoprolol) and placebo treated subjects in Example 1. The change in body weight is given in percentage compared to baseline (mITT population). The data points for treatment and placebo were recorded on the same day during clinic visits. Solid line: placebo; dashed line (Treatment): Tesomet.

FIG. 2: Comparison of change in waist circumference in Tesomet and placebo treated subjects in Example 1. The change in body waist circumference is given in percentage compared to baseline (mITT population). The data points for treatment and placebo were recorded on the same day during clinic visits. Solid line: placebo; dashed line (Treatment): Tesomet.

FIG. 3a: Treatment with Tesomet compared to placebo resulted in a statistically significant difference in the number of responders with a ≥5% body weight reduction from baseline to 24 weeks during the double-blind treatment period and this effect was maintained following an additional 24 weeks of open-label Tesomet treatment. Patients treated with placebo during the first 24 week double-blind period of the study and that then received 24 weeks of Tesomet treatment during the open-label extension period also showed a marked improvement in the number of responders with ≥5% body weight reduction from baseline.

FIG. 3b: Treatment with Tesomet compared to placebo resulted in a significant difference in the number of responders with a ≥10% body weight reduction from baseline to 24 weeks during the double-blind treatment period and this effect remained high following an additional 24 weeks of open-label Tesomet treatment. Patients treated with placebo during the first 24 week double-blind period of the study and that then received 24 weeks of Tesomet treatment during the open-label extension period also showed a marked improvement in the number of responders with ≥10% body weight reduction from baseline.

FIG. 4: Tesomet treatment resulted in clinically meaningful reductions in HbA1c levels in patients with Type-2 diabetes after 24 and 48 weeks of treatment, whereas no effect was seen on normoglycemic patients.

FIG. 5: Treatment with Tesomet compared to placebo resulted in a statistically significant and clinically meaningful reduction in body weight from baseline to 24 weeks of treatment and this effect was maintained following an additional 24 weeks of open-label Tesomet treatment. Patients treated with placebo during the first 24 weeks of the study and that then received 24 weeks of open-label Tesomet treatment also showed clinically meaningful body weight reductions from baseline.

FIG. 6: Treatment with Tesomet compared to placebo resulted in a clinically meaningful reduction in fat mass from baseline to 24 weeks of treatment and this effect was maintained following an additional 24 weeks of open-label Tesomet treatment. Patients treated with placebo during the first 24 weeks of the study and that then received 24 weeks of open-label Tesomet treatment also showed clinically meaningful reductions in fat mass from baseline.

FIG. 7: Patients treated with Tesomet during the 24 week double-blind phase followed by an additional 24 weeks of open-label Tesomet treatment showed evidence of increased lean tissue mass during the open label extension. During the double-blind phase, the Tesomet treated patients had lost lean mas. In contrast, patients receiving placebo during the 24 week double-blind phase followed by 24 weeks of open-label Tesomet treatment showed evidence of decreased lean tissue mass.

### DETAILED DESCRIPTION

HO typically in occurs in patients with tumors and lesions in the medial hypothalamic region. Hypothalamic dysfunction can lead to hyperinsulinemia and leptin resistance. These patients have often suffered damage to the hypothalamus. Damage to the hypothalamus has long been known to promote excessive eating (hyperphagia) and weight gain, termed “hypothalamic obesity.” This form of weight gain is often not responsive to diet and exercise.

Body Mass Index (BMI) is a value derived from the mass (weight) and height of a person. The BMI is defined as the body mass divided by the square of the body height, and is universally expressed in units of kg/m². In one aspect, the present disclosure relates to a method for reducing or maintaining BMI in Hypothalamic Obesity patients.

The terms “subject” and “patient” are used interchangeably herein.

People are generally considered overweight or pre-obese if the BMI is between 25 and 30 and obese if the BMI is over 30. Morbidly obese subjects have a BMI over 35.

In one embodiment the subject has a BMI above 25 kg/m², such as above 30 kg/m², for example above 35 kg/m², such as above 40 kg/m².

In one embodiment the subject has a BMI above 30 kg/m².

In one embodiment the subject has a BMI above 35 kg/m².

Tesofensine is preferably administered to a subject in need thereof once a day. However, in certain embodiments, Tesofensine may be administered more than once a day, such as twice a day or alternatively less than once a day, such as once every second or third day depending on the specific formulation and concentration of the individual components of the composition. The subject treated is preferably a human, such as an adult human aged 18 or older.

In order to approve an ordinary weight loss product, the FDA has the following requirements:
A statistically significant difference in ≥5% in body weight loss from baseline between active- and placebo-treated patients.
At least 35% of patients in the active treatment group to achieve ≥5% in body weight loss from baseline.
The proportion of patients with ≥5% in body weight loss in the active group to be at least twice that of the placebo group

These requirements do not necessarily apply to a rare disease like hypothalamic obesity with intractable obesity that is resistant to lifestyle modifications and standard weight loss treatments

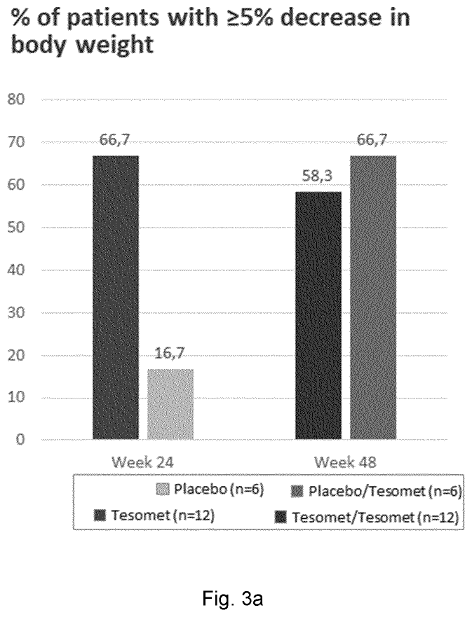

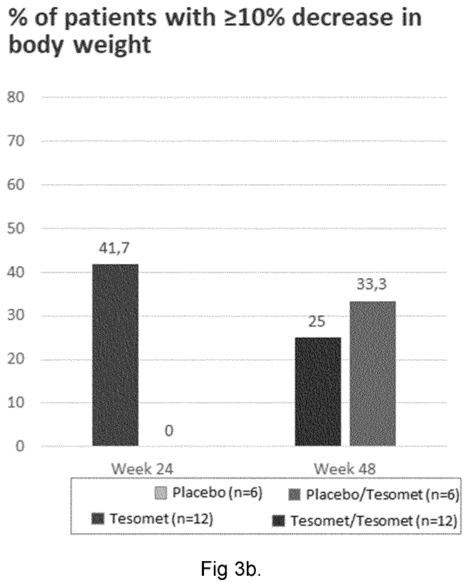

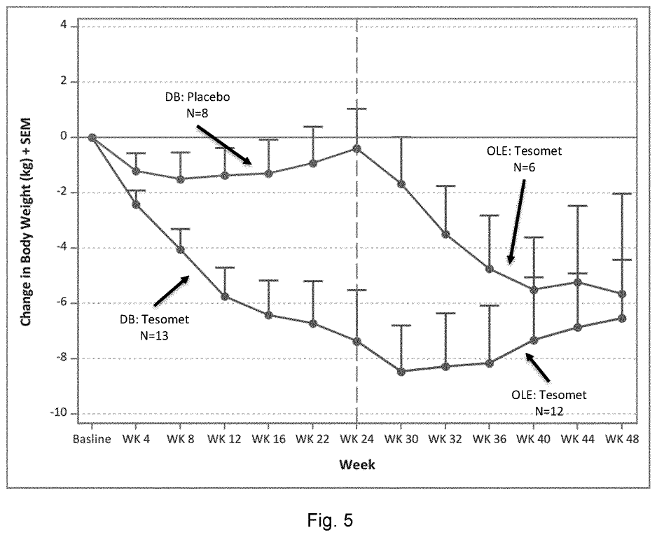

As demonstrated in FIG. 3a approximately ⅔ of the patients experienced at least 5% weight loss after 24 weeks of treatment. 33-41% of the patients experienced at least 10% weight loss thus exceeding the FDA requirements significantly (FIG. 3b). It is also demonstrated that the weight loss is maintained following the first half year of tesofensine therapy (FIG. 5). It is very common for weight loss therapies to have a temporary effect.

In one embodiment, the treatment as described herein leads to an alleviation or improvement of pre-diabetic or diabetic complications.

Type 2 diabetes is a metabolic disorder that is characterized by hyperglycemia in the context of insulin resistance and a relative lack of insulin. Type 2 diabetes makes up about 90% of cases of diabetes, with the other 10% due primarily to diabetes mellitus type 1 and gestational diabetes. Obesity is thought to be the primary cause of type 2 diabetes in people who are genetically predisposed to the disease.

Pre-diabetes is used interchangeably herein with intermediate hyperglycemia. Intermediate hyperglycemia is a biochemical state in which a person has glucose levels above the normal range, but does not yet meet the criteria for a diagnosis of diabetes. The primary aim of management of intermediate hyperglycaemia is to prevent progression to diabetes.

A pre-diabetic subject may have one or more of impaired fasting glycaemia (IFG) and/or impaired glucose tolerance (IGT) and/or elevated glycated haemoglobin (HbA_{1c}) levels.

Weight loss can prevent progression of pre-diabetes into diabetes and can also markedly improve clinical symptoms of type 2 diabetes. Thus, weight loss is an attractive treatment strategy for pre-diabetic subjects and subjects suffering from type 2 diabetes.

In one embodiment the patient suffering from Hypothalamic Obesity is obese and may be pre-diabetic. In one embodiment the patient suffers from type 2 diabetes.

The WHO diabetes diagnostic criteria are shown in the table below.

| | 2 hour | | Fasting | | | |
|---|---|---|---|---|---|---|
| | glucose* | | glucose | | HbA_{1c} | |
| | mmol/l | | mmol/l | | mmol/mol | |
| Condition | (mg/dl) | | (mg/dl) | | (DCCT %) | |
| Normal | <7.8 | (<140) | <6.1 | (<110) | <42 | (<6.0) |
| Impaired | <7.8 | (<140) | ≥6.1 (≥110) & | | 42-46 | (6.0-6.4) |
| fasting | | | <7.0 (<126) | | | |
| glycaemia | | | | | | |
| Impaired | ≥7.8 | (≥140) | <7.0 | (<126) | 42-46 | (6.0-6.4) |
| glucose | | | | | | |
| tolerance | | | | | | |
| Diabetes | ≥11.1 | (≥200) | ≥7.0 | (≥126) | ≥48 | (≥6.5) |
| mellitus | | | | | | |

*Venous plasma glucose 2 hours after ingestion of 75 g oral glucose load

The HO patients benefitting from treatment with the composition of the present disclosure may also suffer from an obesity-associated disorder or condition, such as one selected from the group consisting of pre-diabetes, diabetes, metabolic syndrome, dyslipidemia, atherosclerosis, drug-induced obesity, overeating disorders, bulimia nervosa, binge eating disorder, compulsive over-eating, impaired appetite regulation, nonalcoholic fatty liver disease (NAFLD) and nonalcoholic steatohepatitis (NASH).

In still further embodiments, the HbA1c in the patient is reduced by at least 3 mmol/mol after 6 months of treatment, such as between 3 and 9 mmol/mol, or between 4 and 8 mmol/mol.

In particular, it is expected that the treatment may result in treatment of fatty liver disease, such as nonalcoholic fatty liver disease (NAFLD) or nonalcoholic steatohepatitis (NASH).

Nonalcoholic fatty liver disease (NAFLD) is a cause of a fatty liver, occurring when fat is deposited in the liver (steatosis) due to other causes than excessive alcohol use. NAFLD is the most common liver disorder in Western industrialized nations. NAFLD is associated with insulin resistance and metabolic syndrome (obesity, combined hyperlipidemia, diabetes mellitus (type II) and high blood pressure). Non-alcoholic steatohepatitis (NASH) is the most extreme form of NAFLD, and is a major cause of cirrhosis of the liver. NASH is a state in which the steatosis is combined with inflammation and fibrosis (steatohepatitis).

In one embodiment, the treatment of the present disclosure results in decreasing liver fat and/or visceral adiposity. Reduction of liver fat and/or visceral adiposity has been shown to be effective in the treatment of fatty liver disorders.

### Tesofensine

The methods described herein comprise administration of an active pharmaceutical ingredient (API) selected from tesofensine or a pharmaceutically acceptable salt thereof.

Tesofensine [(1R,2R,3S,5S)-3-(3,4-dichlorophenyl)-2-(ethoxymethyl)-8-methyl-8-azabicyclo[3.2.1]octane] is a centrally acting triple monoamine re-uptake inhibitor (MRI) with intrinsic inhibitory activity on noradrenaline, serotonin and dopamine transporter function. When corrected for placebo and diet effects, long-term Tesofensine treatment produces a weight loss of about 10% in obese patients, which in general is twice as much as that achieved by currently marketed anti-obesity drugs.

The chemical structure of Tesofensine is:

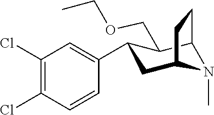

Preclinical and clinical data suggest that appetite suppression is an important mechanism by which Tesofensine exerts its robust weight-reducing effect. In addition, Tesofensine has also been demonstrated to increase nocturnal energy expenditure in human subjects. These findings have recently been corroborated and extended in preclinical settings, demonstrating that Tesofensine induces a robust and sustained weight loss in a rat model of diet-induced obesity (DIO) of which the long-lasting reduction in body weight is caused by appetite suppression with a gradual increase in energy expenditure. Notably, the hypophagic effect of Tesofensine in DIO rats is critically dependent on stimulated α1 adrenoceptor activity, and to a less extend dopamine D1 receptor function, indicating that enhancement of central noradrenergic and dopaminergic neurotransmission constitute important mechanisms underlying the robust appetite-suppressing effect of Tesofensine.

Overall, chronic Tesofensine treatment is associated with minor adverse events, and with minimal cardiovascular effects, suggesting that Tesofensine may generally be a well-tolerated long-term treatment for obesity. However, dose-dependent elevations in heart rate and significant increases in blood pressure have been reported in obese individuals. The long-term implications of such Tesofensine-induced cardiovascular effects are not known and can potentially play a role in the benefit/risk evaluation of patients treated with Tesofensine.

The dosage preferably results in a Tesofensine plasma or serum concentration of 5 to 15 ng/ml at steady state, such as 7-13 ng/mL. It is expected that such plasma level results in weight loss, loss of body fat, or reduction in waist circumference.

For maintenance of body weight, the dosage preferably results in a Tesofensine plasma concentration of 3 to 6 ng/ml at steady state.

### Beta Blockers

The present disclosure involves the use of beta blockers in certain embodiments. The beta blocker may be any conventional beta blocker known in the art. Preferably, the beta blocking drug is selected from the following groups of compounds, which groups of compounds are known in the art and may be commercially available under different brand names, or may be obtained as described in the literature.

In one embodiment, the pharmaceutical composition comprises an extended release (ER) composition of a beta blocker. In one embodiment, the pharmaceutical composition comprises an extended release (ER) composition of a beta blocker and an immediate release (IR) composition of a beta blocker.

In one embodiment, the beta blocker in the ER composition is the same beta blocker as in the IR composition.

In one embodiment, the pharmaceutical composition comprises ER Metoprolol and IR Metoprolol.

As used herein, the term “ER Metoprolol” refers to an extended release (ER) composition of Metoprolol, or a pharmaceutically acceptable salt thereof.

As used herein, the term “IR Metoprolol” refers to an immediate release (IR) composition of Metoprolol, or a pharmaceutically acceptable salt thereof.

### Non-Selective Beta Blockers

In one embodiment, the beta blocker is a non-selective beta blocker.

Examples of non-selective beta blockers include alprenolol, amosulalol, bucindolol, carteolol, levobunolol, mepindolol, metipranolol, nadolol, oxprenolol, penbutolol, pindolol, propranolol, sotalol and timolol.

In one embodiment, the beta blocker is selected from the group consisting of alprenolol, amosulalol, bucindolol, carteolol, levobunolol, mepindolol, metipranolol, nadolol, oxprenolol, penbutolol, pindolol, propranolol, sotalol, timolol and pharmaceutically acceptable salts thereof.

### Beta 1-Selective Beta Blockers

In another embodiment, the beta blocker is a beta 1-selective beta blocker.

Examples of beta 1-selective beta blockers include acebutolol, atenolol, betaxolol, bisoprolol, esmolol, landiolol, metoprolol and nebivolol.

In one embodiment, the beta blocker is selected from the group consisting of acebutolol, atenolol, betaxolol, bisoprolol, esmolol, landiolol, metoprolol, nebivolol and pharmaceutically acceptable salts thereof.

In a particular embodiment, the beta blocker is metoprolol or a pharmaceutically acceptable salt thereof.

### Mixed Alpha and Beta Blockers

In a still further embodiment, the beta blocker is a mixed alpha and beta blocker.

Examples of mixed alpha and beta blockers include carvedilol, celiprolol and labetalol.

In one embodiment, the beta blocker is selected from the group consisting of carvedilol, celiprolol, labetalol and pharmaceutically acceptable salts thereof.

In a particular embodiment, the beta blocker is carvedilol or a pharmaceutically acceptable salt thereof.

### Beta 2-Selective Beta Blockers

In a still further embodiment, the beta blocker is a beta 2-selective beta blocker.

One example of a beta 2-selective beta blocker is butaxamine.

In one embodiment, the beta blocker is butaxamine or a pharmaceutically acceptable salt thereof.

### Pharmaceutically Acceptable Salts

Examples of pharmaceutically acceptable salts include, without limitation, the non-toxic inorganic and organic acid addition salts such as the hydrochloride, the hydrobromide, the nitrate, the perchlorate, the phosphate, the sulphate, the formate, the acetate, the aconate, the ascorbate, the benzene-sulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate, the phthalate, the salicylate, the sorbate, the stearate, the succinate, the tartrate, the toluene-p-sulphonate, and the like. Such salts may be formed by procedures well known and described in the art.

Examples of pharmaceutically acceptable cationic salts of an API include, without limitation, the sodium, the potassium, the calcium, the magnesium, the zinc, the aluminium, the lithium, the choline, the lysinium, and the ammonium salt, and the like, of an API containing an anionic group. Such cationic salts may be formed by procedures well known and described in the art.

In the context of this disclosure the “onium salts” of N-containing compounds are also contemplated as pharmaceutically acceptable salts. Preferred “onium salts” include the alkyl-onium salts, the cycloalkyl-onium salts, and the cycloalkylalkyl-onium salts.

In one embodiment of the present disclosure, Tesofensine is selected from the free base, the citrate salt and the tartrate salt.

Suitable pharmaceutically acceptable salts of metoprolol include any of the salts mentioned herein and preferably include the tartrate, succinate, fumarate or benzoate salts and especially the succinate salt. The S-enantiomer of metoprolol or a salt thereof, particularly the benzoate salt or the sorbate salt, may also be used.

### Pharmaceutical Composition

The dosage of Tesofensine is 0.1 to 1.5 mg, optionally in combination with a beta blocker. When administered with a beta blocker, the two active ingredients may be formulated in one formulation or may be given as two separate entities.

In one embodiment, the pharmaceutical composition comprises:
a. a first composition comprising an extended release (ER) composition of an active pharmaceutical ingredient (API) selected from the beta-blocker or a pharmaceutically acceptable salt thereof, and
b. a second composition comprising an active pharmaceutical ingredient (API) selected from Tesofensine or a pharmaceutically acceptable salt thereof.

Optionally, the pharmaceutical composition may further comprise
c. a third composition comprising an immediate release (IR) composition of an active pharmaceutical ingredient (API) selected from a beta blocker or a pharmaceutically acceptable salt thereof.

In one aspect, the disclosure concerns a pharmaceutical composition comprising said first composition, second composition and third composition. In one embodiment, said pharmaceutical composition comprises no more than 1.5 mg, such as no more than 1 mg, of Tesofensine, or a pharmaceutically acceptable salt thereof; and 5 to 100 mg of ER beta blocker, such as Metoprolol; and 1 to 25 mg of IR beta blocker, such as Metoprolol.

The beta blocker may for example be metoprolol or carvedilol or pharmaceutically acceptable salts thereof. These include the phosphate, succinate, maleate, sulfate, glutarate, lactate, benzoate, and mandelate salts.

The in vitro bio-dissolution profile (as determined by USP Type II apparatus, rotating paddle, with 500 mL of Phosphate buffer at pH 7.4, 37° C. set at rotating speed of 50 rpm) of the beta blocker is preferably as in table 1.

### Table 1. In Vitro Bio-Dissolution Profile of Extended Release Beta Blocker.

| | |
|---|---|
| Dissolution time | Range |

| | | |
|---|---|---|
| 1 | hour | 10-35% |
| 4 | hours | 25-45% |
| 8 | hours | 45-65% |
| 20 | hours | >80% |

It is desirable that release from the extended release formulation starts without delay and that the release rate for a once daily formulation is substantially linear over 16-24 hours, such as for about 20 hours.

For example, the combined in vitro bio-dissolution profile of metoprolol preferably has a dissolution profile lying within one or more of the release ranges in table 2 for different metoprolol IR: ER ratios at various time points (as determined by USP Type II apparatus, rotating paddle, with 900 mL of Phosphate buffer at pH 7.4, 37° C. set at rotating speed of 75 rpm).

TABLE 2
Combined in vitro bio-dissolution profile of metoprolol.

| | | Calculated | | Calculated | | |
|---|---|---|---|---|---|---|
| | | dissolution | Disso- | dissolution | Disso- | |
| Disso- | | 10 mg | lution | 25 mg | lution | |
| lution | | IR + 100 mg | ranges | IR + 100 mg | ranges | Overall |
| time | | ER | (10:100) | ER | (25:100) | range |
| 1 | hour | 13% | 10-20% | 23% | 20-30% | 10-30% |
| 4 | hours | 29% | 20-40% | 38% | 30-50% | 20-50% |
| 8 | hours | 53% | 40-65% | 58% | 50-70% | 40-70% |
| 24 | hours | 88% | >80% | 90% | >80% | >80% |
| 1 | hour | 13% | 10-20% | 23% | 20-30% | 10-30% |
| 4 | hours | 29% | 20-40% | 38% | 25-50% | 20-50% |
| 8 | hours | 53% | 40-65% | 58% | 40-70% | 40-70% |
| 20 | hours | 88% | >80% | 90% | >80% | >80% |

In general the tesofensine of the composition is dissolved within ½-1 hour. The in vitro dissolution profile with tesofensine under the conditions above is at least 80% of the API within 45 minutes.

Many physiological factors influence both the gastrointestinal transit time and the release of a drug from a controlled release dosage form, and thus influence the uptake of the drug into the systemic circulation. A sustained-release dosage form should release the beta blocker at a controlled rate such that the amount of active ingredient available in the body to treat the condition is maintained at a relatively constant level over an extended period of time. The release of an active ingredient from a controlled release dosage form is generally controlled by diffusion through a coating.

It is likewise important that part of the beta blocker is released rapidly so that a therapeutically effective level of the beta blocker is reached rapidly.

In one embodiment, the pharmaceutical composition is in form of a pharmaceutical dosage form, such as a tablet or a capsule. In one embodiment, the pharmaceutical composition is formulated as a dosage unit. In one embodiment, the pharmaceutical composition is formulated as a once daily dosage unit.

In one aspect, the present invention concerns a kit of parts for use in the treatment of hypothalamic obesity in a subject, wherein said kit of parts comprises at least two separate unit dosage forms (A) and (B), wherein
(A) comprises tesofensine or a pharmaceutically acceptable salt thereof; and
(B) comprises a beta blocker, or a pharmaceutically acceptable salt thereof;
wherein (A) and (B) are administered simultaneously, sequentially or separately to the subject. In one embodiment the beta blocker of (B) is metoprolol or a pharmaceutically acceptable salt thereof.

### Similarity Factors

Similarity factor (f2) is a recognized method for the determination of the similarity between the dissolution profiles of a reference and a test compound. Similarity factor (f2) is a logarithmic transformation of the sum of squared error. The similarity factor (f2) is 100 when the test and reference profiles are identical and approaches zero as the dissimilarity increases. The similarity factor has also been adapted to apply to the determination of the similarity between the dissolution profiles of a reference and test compound as they relate to modified release formulations, such as those exemplified herein.

The f2 similarity factor has been adopted in the SUP AC guidelines and by the FDA guidance on dissolution testing of immediate release dosage forms (FDA Guidance for Industry, Dissolution Testing of Immediate Release Solid Oral Dosage Forms, FDA, (CDER), August 1997 (Dissolution Tech. 4, 15-22, 1997)).

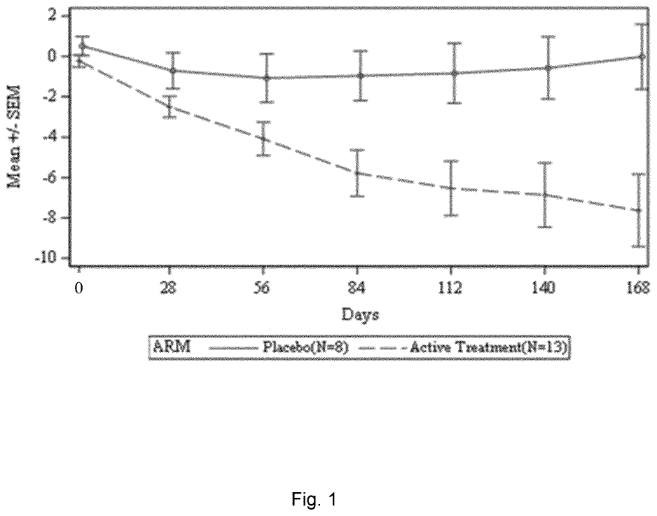

Preferably the pharmaceutical composition has a beta blocker in vitro dissolution profile generated using the USP Type II apparatus, rotating paddle method as described herein with a similarity factor (f2) between 50 and 100 when calculated using one of the examples from FIG. 1 or FIG. 3 in WO 2016/138908 as the reference profile.

### API Amounts and Ratios in Pharmaceutical Composition

### Amount of Beta Blocker

In one embodiment, the pharmaceutical composition as used herein comprises a beta-blocker, or a pharmaceutically acceptable salt thereof. In one embodiment, the pharmaceutical composition as used herein is administered in combination with a beta-blocker, or a pharmaceutically acceptable salt thereof.

The ratio of extended release beta blocker, such as metoprolol, to immediate release beta blocker may be 75-95:25-5. Suitably, the beta blocker, such as metoprolol, in a pharmaceutical composition is approximately an 80:20 ratio of extended release to immediate release amounts, i.e. the ratio of ER Metoprolol/IR Metoprolol is about 4:1 by weight. In another embodiment, the beta blocker, such as metoprolol, is in an approximate 90:10 or 100:10 ratio of extended to immediate release amounts. In one embodiment, the ratio of ER/IR beta blocker, such as ER Metoprolol/IR Metoprolol is about 95:5. In still another embodiment, the ratio is approximately 80:20 or 75:25. Explained differently, for a unit dosage form, such as a tablet, containing 40 mg beta blocker, such as metoprolol, the beta blocker may be present in an amount of about 30 mg in the extended release phase and about 10 mg in the immediate release phase. For a unit dosage form comprising 22 mg beta blocker, such as metoprolol, the beta blocker ER may be present in an amount of 20 mg and the beta blocker IR may be present in an amount of 2 mg. For example, in one embodiment, the ratios of extended release to immediate release phase represent the proportional amount of each layer in a bi-layer dosage form. In another embodiment, the ratios represent the amount of metoprolol in the extended release intragranular component versus the immediate release extragranular component of a single layer dosage form. The ratios and amounts mentioned in the current paragraph apply well to metoprolol as the beta-blocker.

In one embodiment, one dosage form comprises an amount of beta blocker, such as metoprolol, of no more than 100 mg, such as about 75 mg, such as about 50 mg, such as about 25 mg such as about 12.5 mg beta blocker.

Preferably one dosage form comprises an amount of beta blocker, such as metoprolol, ER of 5-200 mg, such as 25-200 mg, such as 5-100 mg API, such as 15-100 mg of API, preferably 15-50 mg, such as 15-40 mg, such as 5-50 mg, such as 5-20 mg, for example about 8 mg, about 20 mg or about 40 mg. In one embodiment, one dosage form comprises an amount of beta blocker, such as metoprolol, ER of no more than 200 mg API, such as no more than 150 mg, such as no more than 100 mg, such as no more than 50 mg, such as no more than 20 mg.

In one embodiment, one dosage form comprises an amount of beta blocker, such as metoprolol, ER of no more than 80 mg, such as about 60 mg, such as about 40 mg, such as about 20 mg, such as about 10 mg.

Other beta-blockers may require lower dosages. In this case one dosage form may comprise an amount of beta blocker, such as carvedilol, ER of 5-40 mg of API, such as 10-20 mg of API, preferably 12-20, for example about 15 mg.

The amount of beta blocker, such as metoprolol, IR per dosage form may be from 1-25 mg API, such as 1-15 mg, for example 3-15 mg, such as 4-10 mg, such as 5-10 mg, such as 1-10 mg, such as 1-5 mg, such as 2-5 mg, for example about 2 mg, about 5 mg, about 10 mg, about 6 mg, or about 8 mg. In one embodiment, the amount of beta blocker, such as metoprolol, IR per dosage form is no more than 25 mg API, such as no more than 20 mg, such as no more than 15 mg, such as no more than 10 mg, such as no more than 5 mg.

In one embodiment, one dosage form comprises an amount of beta blocker, such as metoprolol, IR of no more than 20 mg, such as about 15 mg, such as about 10 mg, such as about 5 mg, such as about 2.5 mg.

The amount of beta blocker as specified herein is based on an the amount of metoprolol tartrate. Other beta blockers, or pharmaceutically acceptable salts thereof, as well as other pharmaceutically relevant salts of metoprolol, or the free base, may also be used in amounts equivalent to the doses of metoprolol tartrate disclosed herein.

In one embodiment, the amount of ER Metoprolol in the pharmaceutical composition is in the range of 1 to 20 mg. In one embodiment, the amount of ER Metoprolol is in the range of 5 to 15 mg. In one embodiment, the amount of ER Metoprolol is 10 mg.

In one embodiment, the amount of IR Metoprolol in the pharmaceutical composition is in the range of 1 to 10 mg. In one embodiment, the amount of IR Metoprolol is in the range of 1 to 5 mg. In one embodiment, the amount of IR Metoprolol is 2.5 mg.

In one embodiment, the combined daily dosis of the beta-blocker are below 125 mg, such as between 5 and 50 mg, between 10 and 30 mg, for example 25 mg, or 12.5 mg. In one embodiment, the combined daily dosis of Metoprolol is less than 25 mg.

### Amount of Tesofensine

The amount of tesofensine per dosage form (such as in the second composition) is generally between 0.01-1 mg API, between 0.125-0.75 mg, such as 0.25-0.5. In one embodiment, the amount of tesofensine per dosage form is no more than 0.75 mg API, such as no more than 0.50 mg, such as no more than 0.250 mg, such as no more than 0.150 mg, such as no more than 0.125 mg. The dose of Tesofensine is based on the amount of the free base, but pharmaceutically relevant salts of Tesofensine may also be used in amounts equivalent to the doses of the free base disclosed herein.

### Amounts of Tesofensine and Beta Blocker

In one embodiment, the ratio of the amount beta blocker, such as metoprolol, to tesofensine is about 200:1. In one embodiment, the ratio of the amount beta blocker, such as metoprolol, to tesofensine is about 100:1. In one embodiment, the ratio of Tesofensine/Metoprolol is about 1:100 by weight. The amount of beta blocker as specified herein is based on an amount of metoprolol tartrate. Other beta blockers, or pharmaceutically acceptable salts thereof, as well as other pharmaceutically relevant salts of metoprolol, or the free base, may also be used in amounts equivalent to the doses of metoprolol tartrate disclosed herein. The amount of Tesofensine is based on the amount of the free base, but pharmaceutically relevant salts of Tesofensine may also be used in amounts equivalent to the doses of the free base disclosed herein.

One dosage form such as a tablet or a capsule may comprise 10-100 mg ER metoprolol, 2.5-25 mg IR metoprolol, and 0.125-1 mg Tesofensine, for example 10-80 mg ER metoprolol, 2.5-20 mg IR metoprolol, and 0.125-1 mg tesofensine, for example 10-60 mg ER metoprolol, 0.25-15 mg IR metoprolol, and 0.125-0.75 mg tesofensine.

One dosage form such as a tablet or a capsule may comprise 10-125 mg ER metoprolol and 0.125-1.5 mg Tesofensine; for example 10-100 mg ER metoprolol and 0.125-1 mg tesofensine, for example 12.5-75 mg ER metoprolol and 0.125-0.75 mg tesofensine.

In one embodiment the beta blocker is metoprolol and the amount of the two APIs in two or three phases of the current dosage form are present in the absolute amounts of table 3.

TABLE 3a
Amounts of Metoprolol ER, and Tesofensine.

| | |
|---|---|
| Metoprolol ER | Tesofensine |

| | | | |
|---|---|---|---|
| 10-125 | mg | 0.125-1.5 | mg |
| 10-100 | mg | 0.125-1 | mg |
| 100 | mg | 1 | mg |
| 75 | mg | 0.75 | mg |
| 50 | mg | 0.50 | mg |
| 25 | mg | 0.25 | mg |
| 12.5 | mg | 0.125 | mg |

TABLE 3b
Amounts of Metoprolol ER, Metoprolol IR and Tesofensine.

| | | |
|---|---|---|
| Metoprolol ER | Metoprolol IR | Tesofensine |

| | | | | | |
|---|---|---|---|---|---|
| 10-100 | mg | 2.5-25 | mg | 0.125-1.5 | mg |
| 10-80 | mg | 2.5-20 | mg | 0.125-1 | mg |
| 5-100 | mg | 2-25 | mg | 0.010-0.250 | mg |
| 5-50 | mg | 2-15 | mg | 0.025-0.250 | mg |
| 5-40 | mg | 2-10 | mg | 0.125-0.250 | mg |
| 5-200 | mg | 1-50 | mg | 0.250 | mg |
| 80 | mg | 20 | mg | 1 | mg |
| 60 | mg | 15 | mg | 0.75 | mg |
| 40 | mg | 10 | mg | 0.50 | mg |
| 30 | mg | 7.5 | mg | 0.375 | mg |
| 20 | mg | 5 | mg | 0.25 | mg |
| 10 | mg | 2.5 | mg | 0.125 | mg |

### Multi-Layer Dosage Form

The extended release phase may be part of a multiple layer tablet, such as a bi or tri-layer dosage form.

In one embodiment, the dosage form comprises a tri-layer dosage unit having an extended release (ER) phase layer with a beta blocker, such as metoprolol or carvedilol, and one immediate release phase layer with a beta blocker, such as metoprolol or carvedilol and another immediate release layer with tesofensine. The ER phase contains a therapeutically effective amount of the beta blocker, such as metoprolol or carvedilol, suitably in granulate form.

In other embodiments, the dosage form is a bi-layer tablet having an ER phase layer with a beta blocker, such as metoprolol or carvedilol and one immediate release layer with both the betablocker (such as metoprolol or carvedilol) and tesofensine.

In other embodiments, the dosage form is a bi-layer tablet having an ER phase layer with a beta blocker, such as metoprolol or carvedilol and one immediate release layer with tesofensine.

### Extended Release Phase

Extended release compositions of beta blockers, such as metoprolol or pharmaceutically acceptable salts of metoprolol are known the art. Non-limiting examples of disclosures of such compositions are found in: WO 2015/004617, WO 2013/084089, WO 2013/030725, WO 2012/052834, WO 2011/143420, WO 2007/09770, WO 2004/069234, WO 2007/110753, WO 2007/029070, WO 2008/012346, and WO 2007/048233. Such extended release compositions typically involve coating the API with an extended release layer that provides an approximated zero-order rate of dissolution of the API.

In one embodiment, the extended release beta blocker, such as metoprolol, is formulated as pellets with pharmaceutically acceptable excipients such as for example binders, film coating polymers, plasticizers, starch, glidants, and disintegrants.

An extended release formulation of carvedilol is also known from U.S. Pat. No. 8,101,209 (Flamel Technologies).

### Inert Core

In some embodiments, the pellets comprise an initial core (inert core) coated with a layer of a beta blocker, such as metoprolol or a metoprolol salt, and further coated with an extended release layer.

As used herein the term initial core refers to a pharmaceutically acceptable core for use in pharmaceutical formulations which core is inert.

In one embodiment there is provided a pharmaceutical composition for extended release comprising pellets coated with a beta blocker, such as metoprolol or a metoprolol salt, wherein each coated pellet comprises a) an inert core comprising at least 50% (w/w) of soluble substance; b) a drug layer comprising the beta blocker, such as metoprolol, which layer covers the inert core; and c) a controlled release layer thereon.

In another embodiment there is provided a pharmaceutical composition wherein the release rate of drug from the pellets part of the pharmaceutical composition comprising a tabletted or encapsulated composition of a multitude of pellets is controlled by the amount or the percentage of the initial core/spheres of the pellets. Preferably, the amount of initial core is from about 15% to about 35% by weight of the controlled release coated pellets before tableting or capsule filling, such as from 20-30%.

In another embodiment the inert core is strengthened by applying a sub-coat on the initial core/sphere. In pharmaceutical compositions wherein pellets comprising the drug are compressed into tablets, the drug pellets are mixed with powder excipients to form a tableting blend. However, the size of the drug coated pellets, often larger than the particle size of the powder excipients, can cause a lack of uniformity of the tableting blend. The preferred uniformity of the tableting blend is such that the average assay of samples of the tableting blend each weighing the equivalent of one tablet lies within the range of 90 to 110 percent of the label dose and the relative standard deviation of the individual assays is less than or equal to 5 percent. The size of the drug pellets is therefore preferably small. When layering a large amount of drug on a small initial core a high degree of stress is exerted on the initial core. This stress may cause attrition particularly when the inert core comprises sugar spheres. To provide a higher degree of physical strength of the inert core without changing the dissolution rate of drug coated pellets, a sub-coat may be applied on an initial core/sphere. Preferably, the amount of the sub-coat is from about 10% to about 40% of the total weight of the sub-coated inert core, more preferably the amount of sub-coat is from about 15% to about 30% of the total weight of the sub-coated inert core, most preferably the amount of sub-coat is about 16% to about 20% of the total weight of the sub-coated inert core.

The inert core of each of the pellets in the pharmaceutical composition may comprise from about 50% to about 100% (per weight) of soluble substance. Preferably the inert core comprises from about 70% to about 90% (per weight) of soluble substances. A preferred initial core comprises a sugar sphere. Sugar spheres have been used in the pharmaceutical industry as excipients. Such sugar spheres used in pharmaceutical compositions generally contain not more than 92% of sucrose, calculated on the dried basis, the remainder consisting of maize starch. Commonly sugar spheres with a core size larger than 500 μm are used. The core size of the inert cores, preferably a sugar sphere, is between about 50 μm and about 500 μm, preferably between about 100 μm and about 400 μm, more preferably from about 250 μm to about 350 μm.

The inert core may comprise an initial core/sphere that is sub-coated with a layer of a plasticized film coating polymer. This sub-coating of an initial core/sphere provides physical strength to the inert core. The film coating polymer may be a hydrophobic or a hydrophilic polymer, or a combination of the two. Suitable film coating polymers can be cellulose derivative polymers or polymethacrylate polymers. Further, hydrophobic polymers or hydrophilic plasticizers, or a combination of several plasticizers can be used to plasticize the film coating polymers. These compounds of the polymeric sub-coat are mixed with solvents prior to their application onto the initial core/sphere. Suitable solvents for use in mixing the polymeric sub-coating compounds are selected from ethanol, isopropyl alcohol, acetone and purified water. For example a mixture of ethanol, acetone and water is preferred for use in mixing a mixture of the preferred sub-coating compounds EthylCellulose (as a film coating polymer), and plasticizers Dibyutyl Sebacate and Polyethylene Glycol (EC, DBS and PEG).

Preferably, the initial core/sphere is a sugar sphere which is sub coated with a mixture of polymers such as cellulose derivatives e.g. ethylcellulose and triethyl citrate, polyethylene glycol, dibutyl sebacate, and dibutyl phthalate, and wherein the sub-coating layer on the initial core/sphere does not alter the release rate of the drug for the pharmaceutical composition. A preferred sub-coat on the sugar spheres comprises ethyl cellulose as a hydrophobic film coating polymer and a combination of two or more plasticizers, at least one hydrophilic and at least one hydrophobic plasticizer. Suitable plasticizers may include for example polyethylene glycols, citrate esters, dibutyl sebacate, diethyl phthalate, and triacetin. Preferred plasticizers are polyethylene glycol and dibutyl sebacate as the hydrophilic and hydrophobic plasticizers respectively. Preferably, the sub-coat comprises about 75% to about 85% ethyl cellulose, about 10% to about 20% polyethylene glycol and about 3% to about 7% dibutyl sebacate by weight of the sub-coat. More preferably, the sub-coat comprises 80% ethyl cellulose, 15% polyethylene glycol and 5% dibutyl sebacate by weight of the sub-coat.

Alternatively, the core may be an insoluble core onto which the active ingredient has been deposited for example by spraying. It may be made from silicon dioxide, glass or plastic resin particles. Suitable types of plastic material are pharmaceutically acceptable plastics such as polypropylene or polyethylene preferably polypropylene. Such insoluble cores may have a diameter in the range of 0.01-2 mm, preferably in the range of 0.05-1.0 mm and more preferably in the range of 0.1-0.7 mm.

### Beta Blockers for Extended Release

In one embodiment, a beta blocker, such as Metoprolol or its acceptable pharmaceutical salt, may be applied on the inert core. No use of “Class 2” solvents (as defined by the FDA) is required to apply the active pharmaceutical ingredient (API), drug, onto the inert core forming a drug coated pellet. The FDA defines “Class 2” solvents as having inherent toxicity. The active ingredient is dispersed in water, preferably together with an acceptable binder excipient such as, but not limited to, polyvinyl pyrrolidone, cellulose derivatives polymers, or starch.

The beta blocker, such as metoprolol may be applied as a dispersion rather than a solution. Therefore it is preferred that the drug substance has physical properties that will allow a high yield in preparing drug coated pellets. Therefore, the drug substance preferably has a particle size distribution such that the d(0.9) value is less than about 80 μm. Preferably, the d(0.9) value for the particle size distribution of the drug substance is less than about 50 μm, more preferably less than about 30 μm. As a result, a concentrated dispersion for application can be produced which may shorten the production time.

The drug coated pellets may comprise from about 40% to about 90% (per weight) of the drug layer, preferably from about 50% to about 80% (per weight), more preferably from about 55% to about 75% (per weight).

Other beta blockers, such as Carvedilol or salts thereof, may be applied in a similar as indicated for Metoprolol.

### Controlled Release Layer

The last layer applied on the pellets is a layer which controls the release of the active pharmaceutical ingredient. Pellets that have been coated with a controlled release layer may have a size between about 200 μm and about 800 μm. Preferably, the controlled release layer coated pellets have a size ranging from about 300 μm to about 700 μm, more preferably from about 400 μm to about 600 μm. In addition, the controlled release layer may comprise water soluble and insoluble components. Such components may be film forming polymers and plasticizers. For example, a film comprising a polymeric layer may be applied onto the drug coated pellets.

In the following three different types of extended release coatings are described.

### First Extended Release Coating

In one embodiment the extended release film coat comprises i) an acrylic polymer ii) a surfactant and iii) sodium stearyl fumarate, wherein the film coat has been deposited from a water containing liquid.

Typically a film coating composition comprises
a) 25 to 35% by weight of an acrylic polymer dispersion
b) 0.1 to 4% by weight of a surfactant
c) 0.1 to 4% sodium stearyl fumarate and
d) a water-containing liquid to 100%.

In one embodiment there is provided film coatings which are suitable for giving extended release. Suitably the acrylic polymer used in this case comprises homogeneous particles wherein the polymer or copolymer has T_{g}<room temperature in aqueous dispersion but has T_{g}>room temperature in the dry state. Suitable polymers comprise acrylic acid and esters thereof particularly the methyl, ethyl, propyl and butyl esters; and methacrylic acid and esters thereof particularly the methyl, ethyl, propyl and butyl esters. Particularly preferred polymers are those provided under the tradenames Eudragit L30DR (Rohm Pharma) or Eudragit FS30D® (Rohm Pharma). Optionally further anti-tacking agents may be required.

Suitably the amount of the acrylic polymer in the film coating composition is in the range of 15 to 50% by weight. Preferably the amount of the acrylic polymer in the film coating composition is in the range of 20 to 40% by weight. More preferably the amount of the acrylic polymer in the film coating composition is in the range of 25 to 35% by weight.

Suitably the surfactant is one of the following: a nonionic surfactant, like sorbitan esters (Span series); polysorbates (Tween series); polyoxyethylated glycol monoethers (like the Brij series); polyoxyethylated alkyl phenols (like the Triton series or the Igepal series); alkyl glucosides (e g dodecylmaltoside); sugar fatty acid esters (e g sucrose laurate); saponins; etc: or mixtures thereof; ampholytic surfactants, like betaines; anionic surfactants, like sulphated fatty alcohols eg sodium dodecylsulphate SDS; sulphated polyoxyethylated alcohols; others like dioctyl sulphosuccinate; bile salts (e g dihydroxy bile salts like sodium deoxycholate, trihydroxy bile salts like sodium glycocholate, etc); fusidates (e g sodium dihydrofusidate); etc cationic surfactants, like ammonium compounds; soaps, fatty acids, and lipids and their salts, like alkanoic acids; (e g octanoic acid, oleic acid); monoglycerides (eg monolein), phospholipids which are neutral or positively or negatively charged (eg dialkyl phosphatidylcholine, dialkyl phosphatidylserine, etc); etc; more preferably the surfactant is a nonionic surfactant. Most preferably the surfactant is nonoxynol 100.

Suitably the amount of the surfactant in the film coating composition is in the range of 0.05 to 8% by weight. Preferably the amount of the surfactant in the film coating composition is in the range of 0.1 to 6% by weight. More preferably the amount of the surfactant in the film coating composition is in the range of 0.5 to 4% by weight.

In a most preferred embodiment the acrylic polymer and the surfactant are provided by Eudragit® NE30D in compositions, a film coats or formulations defined previously.

Suitably the amount of the sodium stearyl fumarate in the film coating composition is in the range of 0.05 to 8% by weight. Preferably the amount of sodium stearyl fumarate in the film coating composition is in the range of 0.1 to 6% by weight. More preferably the amount of sodium stearyl fumarate in the film coating composition is in the range of 0.5 to 4% by weight.

Suitably the water-containing liquid comprises water and a water miscible organic liquid for example lower alkanols e.g. ethanol, propanol or isopropanol. From a safety point of view is preferred that the proportion of the organic is kept to a minimum but small amounts are tolerable for example in the range of 0 to 20% by volume. Preferably the liquid is water.

The film-coating composition is particularly suitable for use as an aqueous film-coating composition wherein the film-coat is applied using water as the liquid. When the liquid is water the latex is preferably a poly(ethylacrylate-co-methylmethacrylate) copolymer, for example Eudragit NE30D® (Rohm Pharma). This process is particularly advantageous as it negates the need to use environmentally unacceptable organic solvents, some of which also present processing problems due to their inflammablility, while also eliminating many of the problems experienced with aqueous coatings described above.

### Second Extended Release Coating

Alternatively, the film may comprise at least one film coating polymer and can be plasticized with one or more plasticizers. These plasticizers may differ from each other in their degree of solubility (hydrophobicity/hydrophilicity). By changing the ratio between the plasticizers and the film coating polymer, or the ratio between the different plasticizers (if more than one is used), one can control the rate of the release of the drug from the pellets. The controlled release layer of the beta blocker ER may comprise a hydrophobic film coating polymer such as for example ethylcellulose and a combination of at least two plasticizers, at least one hydrophilic and one hydrophobic plasticizer, for example polyethylene glycol and dibutyl sebacate. Preferably, the ratio of hydrophobic to hydrophilic plasticizer in the controlled release layer of the pharmaceutical composition is from 3:1 to 1:3, more preferably the ratio is 1:1.

Furthermore, the controlled release layer may comprise at least about 70% water insoluble compounds (per weight of the controlled release layer). Preferably, the controlled release layer comprises at least about 80% and more preferably at least about 90% water insoluble compounds (per weight of the controlled release layer). Suitable water insoluble compounds are for example cellulose derived polymers. These controlled release layer compounds are mixed with solvents prior to their application onto the drug coated pellets. Suitable solvents for use in mixing the controlled release layer compounds are selected from ethanol, isopropyl alcohol, acetone and purified water. A mixture of ethanol, acetone and water is preferred for use in mixing the controlled release layer compounds especially where the controlled release layer compounds are a mixture of ethylcellulose, dibutyl sebacate and polyethylene glycol.

The method of preparing the beta blocker ER component may comprise sub-coating an initial core/sphere forming an inert core. Sub-coating an initial core/sphere comprises mixing a film coating polymer with one or more plasticizers in a solvent forming a coating mixture. Such mixture may be a solution, suspension or slurry for applying a coating layer on a surface. The coating mixture is applied to the initial core/sphere forming a sub-coated initial core/sphere which is used as an inert core. The film coating polymer may be a hydrophobic or a hydrophilic polymer, or a combination of the two. Suitable film coating polymers can be cellulose derivative polymers or polymethacrylate polymers, preferably ethylcellulose. The amount of ethylcellulose is preferably from about 75% to about 85% more preferably about 80% of the total amount of the weight of the sub-coat. Further, hydrophobic polymers or hydrophilic plasticizers, or a combination of several plasticizers can be used to plasticize the film coating polymers. These compounds of the polymeric sub-coat are mixed with solvents prior to their application onto the initial core/sphere. Suitable solvents for use in mixing the polymeric sub-coating compounds are selected from ethanol, isopropyl alcohol, acetone and purified water. A mixture of ethanol, acetone and water is preferred for use in mixing the polymeric sub-coating compounds.

Suitable plasticizers for use in sub-coating an initial core/sphere are selected from polyethylene glycol, dibutyl sebacate, and dibutyl phthalate. Preferred plasticizers are polyethylene glycol and dibutyl sebacate as the hydrophilic and hydrophobic plasticizers respectively. Preferred amounts of plasticizers used in the method are about 10% to about 20% polyethylene glycol and 3% to about 7% dibutyl sebacate by weight of the sub-coat. More preferably, about 15% polyethylene glycol and 5% dibutyl sebacate as plasticizer.

For the extended release coat, the amount of ethylcellulose is preferably from about 75% to about 85% more preferably about 80% of the total amount of the weight of the coat. Suitable plasticizers for use in the ER-coating are selected from polyethylene glycol, dibutyl sebacate, and dibutyl phthalate. Preferred plasticizers are polyethylene glycol and dibutyl sebacate as the hydrophilic and hydrophobic plasticizers respectively. Preferred amounts of plasticizers used in the method are about 5% to about 20% polyethylene glycol and dibutyl sebacate by weight of the ER-coat. More preferably, about 10% polyethylene glycol and 10% dibutyl sebacate as plasticizer.

In one embodiment, a metoprolol ER tablet comprises components according to table 4.

TABLE 4
Metoprolol ER tablet.

| | | |
|---|---|---|
| | | Percent total |
| Material | Weight | pellet weight |

Sub-coated pellets

| | | |
|---|---|---|
| Sugar spheres (250-355 μm) | 598.00 | 22.3 |
| Ethyl cellulose 7 cps | 92.00 | 3.4 |
| Polyethylene glycol 400 | 17.25 | 0.6 |
| Dibutyl sebacate | 5.75 | 0.2 |

Drug layer

| | | |
|---|---|---|
| Metoprolol succinate | 1092.50 | 40.9 |
| Polyvinyl pyrrolidone | 276 | 10.3 |
| povidone (PVP K-30) | | |

Controlled release film layer

| | | |
|---|---|---|
| Ethyl cellulose 100 cps | 473.8 | 17.7 |
| Polyethylene glycol 400 | 59.23 | 2.2 |
| Dibutyl sebacate | 59.23 | 2.2 |

In a preferred method of preparing the beta blocker ER part of the composition, the method comprises the following steps; a) providing sugar spheres as initial cores; b) coating the sugar spheres with a sub-coat comprising mixing a film of a hydrophobic polymer, a soluble (hydrophilic) plasticizer, and an insoluble (hydrophobic) plasticizer with a solvent mixture of e.g. acetone, ethanol 95%, and water and spraying the mixture onto the sugar spheres to create a sub-coat on the sugar spheres resulting in an inert core; c) coating the sub-coated sugar spheres (inert cores) with a drug layer comprising mixing the drug, such as metoprolol succinate, and a binder, preferably povidone (PVP K-30) with preferably water, forming an aqueous dispersion and applying the dispersion onto the sub-coated pellets (inert cores) forming drug coated pellets; d) applying a third layer on the drug coated pellets comprising dissolving a hydrophobic film coating polymer, an hydrophilic plasticizer and an hydrophobic plasticizer in a solvent mixture of e.g. acetone, ethanol 95%, and water forming a mixture and spraying the mixture onto the drug coated pellets to create controlled release drug coated pellets; e) mixing the controlled release drug coated pellets with a powder mixture of one or more excipients forming a final blend; f) compressing the final blend into tablets or filling the final blend into capsules; and g) optionally film coating the tablets for cosmetic purposes.

In this method the hydrophobic polymer is preferably ethyl cellulose (EC), the soluble/hydrophilic plasticizer is preferably polyethylene glycol (PEG), and the insoluble/hydrophobic plasticizer is preferably dibutyl sebacate (DBS). Further, in preparing a mixture for coating the sugar spheres with a sub-coat, and the drug coated pellets with a controlled release layer, ethyl cellulose is preferably first dissolved in acetone and ethanol 95%, then PEG and DBS are added, followed by adding water and mixing the solution till it is homogenized. Preferably, the spraying of a solution or dispersion onto sugar spheres or drug coated pellets in the method uses a fluidized bed coater with a Wurster insertion. Furthermore, the binder, used in coating the sub-coated sugar spheres with a drug layer, facilitates binding of the drug to the inert core of sub-coated sugar spheres. Moreover, in this method the ratio of powder mixture to controlled release drug coated pellets in the final tableting blend is preferably from about 20% to about 60% (by weight), more preferably from about 30% to about 50% (by weight), most preferably from about 35% to about 45% (by weight). As a result a uniform final tableting blend and tablets are produced.

### Third Extended Release Coating

An extended release phase may comprise at least one high viscosity hypromellose (HPMC) ingredient. HPMC is a water soluble matrix-forming polymer used to provide an extended release effect of metoprolol. The viscosity of the HPMC used in the ER phase may be up to 100.000 centipoise such as in the range of about 3500-6000 cps.

An extended release layer with a therapeutically effective amount of a beta blocker, such as metoprolol or carvediol, can be made with high viscosity hypromellose alone.

In other embodiments, the extended release layer comprises a therapeutically effective amount of a beta blocker, such as metoprolol or carvediol, at least one high viscosity hypromellose, at least one binding agent, a low viscosity hypromellose, at least one modified starch, and optionally one or more other pharmaceutically acceptable intragranular components including but not limited to a second pharmaceutically acceptable active ingredient, other pharmaceutically acceptable excipients and/or adjuvants. In one embodiment, the ratio of high-viscosity hypromellose to low viscosity hypromellose is about 3.3 to about 0.85. In another embodiment the ratio of high to low is about 3:1.

Suitably, the viscosity of the low viscosity hypromellose is in the range of about 10-30 centipoises. In another embodiment the low viscosity is about 15 centipoises.

The amount of at least one binding agent in the extended release phase of a bilayer tablet may be from about 0.5% to about 3% w/w. In one embodiment there are at least two binding agents present in the ER phase. Suitably the amount of at least one modified starch in the extended release phase of the bilayer tablet is from about 0.5% to about 3% w/w. In one embodiment, the amount of modified starch is about 1% w/w of the ER phase. In one embodiment there are at least two modified starches present in the ER phase. Suitably, the modified starch is pre-gelatinized.

Suitably, the amount of the high viscosity hypromellose present in the extended release phase is from about 3%> to about 7%> of the extended release phase formulation weight. In another embodiment, the amount of high viscosity hypromellose is from about 4% to about 6%. In still other embodiments, an amount of >20% hypromellose is used in the extended release phase.

In yet another embodiment the amount of high viscosity HPMC is present in an amount of about 5% w/w extended release phase formulation weight.

Suitably, the amount of the low viscosity hypromellose present in the extended release phase is from about 0.5% to about 3% of the extended release phase formulation weight. In another embodiment, the amount of low viscosity hypromellose is from about 1% to about 2% of the extended release phase formulation weight.

Alternatively, the total amount of cellulosic derivatives of HPMC present in the ER granulate range from about 3% to about 10% by weight of the total amount of extended release components. This encompasses both the high and the low viscosity HPMC's.

In one embodiment the ER phase comprises metoprolol, povidone, pre-gelatinized corn starch, and a high and low viscosity HPMC.

In one embodiment the ER phase comprises carvedilol, povidone, pre-gelatinized corn starch, and a high and low viscosity HPMC.

### Tablets and Capsules

The film coated beads or spheres may be provided in sachets or formulated as a capsule, for example a hard gelatin capsule, or compressed to form tablets using known methods with the optional addition of other pharmaceutically acceptable additives and with the addition of the beta blocker IR and tesofensine components herein described. Coated beads to be compressed into a tablet are obtained by conventional techniques known to those skilled in the art.

Also, during this process suitable other agents can be added. For example, during the tabletting step suitable fillers, e.g. microcrystalline cellulose, lactose monohydrate, talc. sodium stearyl fumarate etc can be utilised to give acceptable compression characteristics of the formulation, e g hardness of the tablet.

These additives can be granulated in one of the conventional granulation methods. However, preferably there is provided a set of additives, for example a powder mixture that can be directly compressed into tablets. Such powder mixture serves as a filler, cushioning, disintegrant, glidant, and lubricant mixture. Furthermore, the ratio of controlled release drug coated pellets to additives in the final (e.g. tableting) blend of the present pharmaceutical composition is of particular importance to prepare a uniform product e.g. tablets.

To prepare a uniform product, preferably at least 50% (by weight) of the powder mixture may have particle sizes between about 30 μm to about 800 μm, preferably from about 80 μm to about 600 μm, more preferably from about 100 μm to about 300 μm. More preferably, at least 65% (by weight) of the powder mixture has particle sizes between about 30 μm to about 800 μm, preferably from about 80 μm to about 600 μm, more preferably from about 100 μm to about 300 μm. Most preferably, at least 80% (by weight) of the powder mixture has particle sizes between about 30 μm to about 800 μm, preferably from about 80 μm to about 600 μm, most preferably from about 100 μm to about 300 μm.

Furthermore, the amount of controlled release drug coated pellets in the final tableting blend is preferably from about 20% to about 60% (by weight) in order to prepare such uniform product. More preferably, the amount of controlled release drug coated pellet in the final tableting blend is from about 30% to about 50% (by weight), most preferably from about 35% to about 45% (by weight).

Suitable powder mixtures comprise, but are not limited to, mixtures of two or more of the following compounds; Starlac® (a spray-dried compound consisting of 85% alpha-lactose monohydrate and 15% maize starch dry matter available from Meggle), Cellactose® (a spray-dried compound consisting of 75% alpha-lactose monohydrate and 25% cellulose powder dry matter available from Meggle), Parteck® (A Directly Compressible Sorbitol available from Merck KGaA), Crospovidone, Silicon Dioxide, Magnesium Stearate, Talc, Zinc Stearate, Polyoxyethylene Stearate, Stearic Acid, sodium stearyl fumarate Cellulose derivatives, microcrystalline cellulose and lactose monohydrate.

If the dosage form is a bi- or tri-layer tablet, the immediate release layer(s) may be compressed directly on a previously partly compressed extended release layer, or alternatively, the extended release layer may be compressed onto previously partly compressed immediate release layer(s).

The compositions can be formulated by conventional methods of admixture such as granulating, blending, filling and compressing. For example, tablets can be produced by a wet granulation process, where the immediate release phase and extended release phase are separately prepared. Suitably, for either the immediate release or extended release phase, the active drug substance and excipients are screened and mixed in a high shear mixer granulator or fluid bed dryer. The blend is granulated by the addition of a granulating solution (typically purified water, disintegration agent dissolved/dispersed in purified water, or drug dissolved/dispersed in purified water or a suitable solvent) sprayed into the high shear mixer granulator or fluid bed dryer. If desired wetting agents e.g., surfactants can be added. The resulting granules (optionally pelletized) are dried usually with residual moisture of 1-5% by tray, fluid bed or microwave drying techniques. The dried granules are milled to produce a uniform particle size, the granules are blended with extragranular excipients as necessary, typically a lubricant and glidant (e.g., magnesium stearate, silicon dioxide). The separately prepared immediate release and extended release granules can then be compressed together using a rotary tablet press (such as a bilayer tablet press) if desired. If the dosage form is a single layer tablet, then the extended release granules are admixed with the immediate release extragranular components and compressed together using a rotary tablet press, etc. These resulting tablets can all be coated in a pan coater typically with a 1-5% aqueous film coat, followed by a wax polishing.

Alternatively tablets can be produced by a direct compression process. Suitably the active drug substance and excipients for the immediate release and extended release phases are separately screened and mixed in a suitable blender e.g., a cone, cube or V-blender. Other excipients are added as necessary, and further blended. The separately prepared immediate release and extended release phases can be combined and compressed together using a rotary tablet press as hereinbefore described. The resulting tablets can be coated in a pan coater.

Tablets can also be prepared by using both methods of wet granulation and direct compression. For example the extended release phase can be prepared by wet granulation as described herein, while the immediate release phase can be prepared by blending the excipients for direct compression. The two phases can then be combined and compressed together as hereinbefore described.

### Immediate Release Phase(s)

The immediate release phase(s) may be prepared by combining a directly compressible commercially available grade of the beta blocker, such as metoprolol, and tesofensine with a lubricant, and one or more disintegrating agents if necessary or desired. Binders and other excipients and/or adjuvants may be included in the immediate release layer(s), also if necessary or desired. The beta blocker and tesofensine in the immediate release layer may be combined with a modified starch such as a pre-gelatinized starch, e.g., corn starch, polyethylene glycol, and a disintegrant, or super disintegrant such as croscarmellose sodium or Explotab®, a binder such as methylcellulose or hypromellose polymer, plasticizer, pigment and a lubricant.

The immediate release phases may comprise two different layers of the beta blocker and tesofensine, respectively. Alternatively, the immediate release phases may be combined into one and the same layer. The immediate release phases may also be formulated into an extragranular phase of a tablet or be granulated into one or two different immediate release granules. For tesofensine, the preferred formulation is a granulation of tesofensine compared to direct compression of tesofensine as the dose is relatively low.

### Monolith Dosage Form

In one embodiment, there is only a single layer tablet having an extended release intra-granular phase and two immediate release extra-granular phases. The extended release phase will be comprised of an intra-granular component of the beta blocker and excipients as described above. These components form the ER granulate. The ER blend could be made into pellets and compressed accordingly with the extra-granular immediate release blend.

A suitable extra-granular component or phase, i.e., the immediate release phases, may be prepared by combining a directly compressible commercially available grade of a beta blocker, such as metoprolol, and tesofensine citrate with a lubricant, and one or more disintegrating agents if necessary or desired. As mentioned above for tesofensine the preferred process is to prepare a granulate of tesofensine before compression. Binders and other excipients and/or adjuvants may be included in the extra-granular phase if necessary or desired. Alternatively, an extra-granular component can be prepared by combining the beta blocker, such as metoprolol, and tesofensine with a modified starch, such as a pre-gelatinized starch, e.g., corn starch, a disintegrant or super disintegrant, such as croscarmellose sodium, a binder and a lubricant.

### Excipients

The present compositions may include components that functions as a binder or binding agent. Suitably, the binding agent may comprise a first binding agent and a second binding agent. Suitable binding agents for use herein include conventional binding agents used in the art such as gelatin, starches, povidone, polymers and cellulose derivatives or combinations thereof.

Suitably, the starch, is of vegetable origin, such as corn (or maize) starch, modified corn starch, wheat starch, modified wheat starch, potato starch, or pre-gelatinized starch e.g., available commercially as Starch 1500 G or Prejel; or a combination of two or more thereof.

If the binding agent includes a cellulosic derivative such as hydroxypropyl cellulose (HPC) (of low to medium viscosity) e.g., as may be available commercially under the brand name Klucel® from the Aqualon division of Hercules Inc., Dow Chemical Company e.g., Klucel GF, Klucel JF, Klucel LF and Klucel EF; microcrystalline cellulose (MCC), carboxymethylcellulose (MC), sodium carboxymethylethyl cellulose; or a combination of two or more thereof. Combinations of a cellulosic derivative with other binding agents noted above are also envisaged. Generally the total amount of cellulosic derivatives present in the granulate are in an amount ranging from about 3% to about 10% by weight of the extended release components. It is recognized in the art that certain cellulosic derivatives, such as hypromellose, will have varying roles in a formulation, depending upon the amount used. For example hypromellose (low or medium viscosity) may function as a binding agent, a coating agent, or as a matrix forming agent.

While a binding agent is present as an intra-granular component, it is recognized that a modest amount of binding agent e.g., up to about an additional 3.0%>-10.0% by weight of the intra-granular binding agent content of the composition, may also be present extra-granularly.

In one embodiment, suitably the starch is pre-gelatinized starch. Pre-gelatinized starch is a starch that has been chemically and/or mechanically processed. Typically pre-gelatinized starch contains 5% of free amylase, 15% of free amylopectin, and 80% unmodified starch. Pre-gelatinized starch may be obtained from corn (or maize), potato or rice starch.

The granulate provides an intimate admixture of a combination of ingredients and may then be mixed with one or more pharmaceutically acceptable extra-granular components of the composition i.e., with any pharmaceutically acceptable ingredient e.g., a diluent, flavor, sweetening agent, binder, disintegrant, glidant, lubricant, anti-adherent, anti-static agent, anti-oxidant, desiccant, or a second pharmaceutically acceptable active agent. It is recognized that these same ingredients may be present both as an intra-granular and as an extra-granular ingredient.

As noted above there are other inactive ingredients that may optionally be employed in relatively small quantities, which include lubricants, flow agents, and binders that facilitate compression.

Suitable disintegrating agents include a non-super disintegrant, a super disintegrant or a combination of both. Suitable non-super disintegrants include conventional disintegrants such as starch (corn or maize), pre-gelatinized starch e.g., Starch 1500 G, clays (e.g. VEEGUM (Vanderbilt Minerals, LLC) or Bentonite (an absorbent aluminium phyllosilicate clay consisting mostly of montmorillonite)), microcrystalline cellulose, cellulose or powdered cellulose. It is recognized in the art, that some excipients may perform more than one role in a given pharmaceutical formulation. For example certain excipients, e.g., starches including pre-gelatinized starch, and microcrystalline cellulose (hereinbefore identified as binding agents) function as both binders and disintegrants.

A “super disintegrant” represents a class of disintegrating agent which may generally be used in lower amounts in pharmaceutical preparations, as compared to conventional disintegrants. Examples of super disintegrants include sodium starch glycolate, the sodium salt of carboxymethyl starch, modified cellulose and cross-linked polyvinyl pyrrolidone. Sodium starch glycolate is available commercially under the trade names Explotab® (Edward Mendell Co. JRS Pharma), Primojel® (Generichem Corp; DFE Pharma) and Tablo® (Blanver, Brazil). An example of modified cellulose includes croscarmellose sodium, the sodium salt of carboxymethyl cellulose. Croscarmellose sodium is available commercially under the trade names AcDiSol® (FMC Corp.), Nymcel ZSX® (Nyma, Netherlands), Primellose® (Avebe, Netherlands), Solutab® (Blanver, Brazil). An example of a cross-linked polyvinyl pyrrolidone includes crospovidone, and is commercially available under the trade names Kollidon CL® or Kollidon CL-M (Basf Corp.), and Polyplasdone XL® (ISP Corp; Ashland). A suitable super disintegrants includes croscarmellose sodium or sodium starch glycolate (e.g. Explotab® (JRS Pharma)) or a combination thereof. A super disintegrant may be used extragranularly, in an amount ranging from about 0.5% to about 5.0% by weight of the composition. Suitable preservative or antimicrobial agents for use include potassium sorbate or a paraben, i.e., one or more hydroxy benzoic acid esters e.g., methyl, ethyl, propyl or butyl, suitably singularly or as mixtures. Parabens are commercially available under the Nipa® brand name, e.g., Nipasept® sodium (Aako BV).

Suitable lubricants include magnesium, calcium or sodium stearate, stearic acid or talc that may be added in suitable amounts. In one embodiment the lubricant is magnesium stearate.

Suitable flow agents include silicon dioxide (e.g. Cab-O-Sil® (Cabot Corporation), Syloid™ (W.R. Grace & Co.)) and colloidal silicon dioxide (Aerosil® (Evonik Resource Efficiency GmbH)), that may be added in an amount from about 0.5% to about 1% by weight.

The compressed tablet may further comprise a film coat e.g., hypromellose or polyvinyl alcohol-part.hydrolised (PVA). Suitably the film coat is a transparent film coat e.g., a dye, although an opaque film coat e.g., as obtained when using a film coat in combination with an opacifier or a pigment such as titanium dioxide or a lake may also be used. For example one commercially available film coat is an Opadry® coating system from Colorcon.

### Items

1. A method for treatment of hypothalamic obesity comprising daily administration of a pharmaceutical composition comprising 0.01 to 1.5 mg tesofensine or a pharmaceutically acceptable salt to a patient suffering from hypothalamic obesity.
2. The method according to item 1, wherein the tesofensine is selected from the free base, the citrate salt, and the tartrate salt.
3. The method according to any of the preceding items, wherein the pharmaceutical composition further comprises a beta blocker or a pharmaceutically acceptable salt thereof.
4. The method according to any one of the preceding items, wherein tesofensine is administered in combination with a beta blocker or a pharmaceutically acceptable salt thereof.
5. The method according to any of the preceding items, wherein the daily dose of the beta-blocker is below 125 mg, such as between 10 and 100 mg, for example below 100 mg, such as 75 mg, 50 mg, 25 mg, or 12.5 mg.
6. The method according to any of the preceding items, wherein the daily dose of tesofensine is below 1.5 mg, such as below 1 mg, for example below 0.75 mg, such as 0.5, 0.25, or 0.125 mg of API.
7. The method according to any of the preceding items, wherein the beta blocker is selected from the group consisting of a beta 1-selective beta blocker, a mixed alpha and beta blocker, a non-selective beta blocker and a beta 2-selective beta blocker.
8. The method according to any of the preceding items, wherein the beta blocker is a beta 1-selective beta blocker, such as a beta 1-selective beta-blocker selected from the group consisting of metoprolol, acebutolol, atenolol, betaxolol, bisoprolol, esmolol, landiolol, nebivolol and pharmaceutically acceptable salts thereof.
9. The method according to any of the preceding items, wherein the beta blocker is a mixed alpha and beta blocker, such as a mixed alpha and beta blocker selected from the group consisting of carvedilol, celiprolol, labetalol and pharmaceutically acceptable salts thereof.
10. The method according to any of the preceding items, wherein the beta blocker is a non-selective beta blocker, such a non-selective beta blocker selected from the group consisting of alprenolol, amosulalol, bucindolol, carteolol, levobunolol, mepindolol, metipranolol, nadolol, oxprenolol, penbutolol, pindolol, propranolol, sotalol, timolol and pharmaceutically acceptable salts thereof.
11. The method according to any of the preceding items, wherein the beta blocker is a beta 2-selective beta blocker, such as butaxamine or pharmaceutically acceptable salts thereof.
12. The method according to any of the preceding items, wherein the beta blocker is metoprolol or a pharmaceutically acceptable salt thereof.
13. The method according to any of the preceding items, wherein the beta blocker is selected from metoprolol succinate and metoprolol tartrate.
14. The method according to any of the preceding items, wherein the beta blocker is carvedilol or a pharmaceutically acceptable salt thereof.
15. The method according to any of the preceding items, wherein the beta blocker is released as an extended release formulation, with a substantially linear release over 16-24 hours after administration.
16. The method according to any of the preceding items, wherein the beta-blocker prevents or alleviates the cardiovascular side-effects of tesofensine.
17. The method according to any of the preceding items, wherein the pharmaceutical composition comprises:
   a. a first composition comprising an extended release (ER) composition of an active pharmaceutical ingredient (API) selected from the beta-blocker or a pharmaceutically acceptable salt thereof,
   b. a second composition comprising an active pharmaceutical ingredient (API) selected from Tesofensine or a pharmaceutically acceptable salt thereof, and optionally
   c. a third composition comprising an immediate release (IR) composition of an active pharmaceutical ingredient (API) selected from a beta blocker or a pharmaceutically acceptable salt thereof.
18. The method according to any of the preceding items, comprising administering 10-100 mg ER metoprolol, 2.5-25 mg IR metoprolol, and 0.125-1 mg tesofensine, for example 10-80 mg ER metoprolol, 2.5-20 mg IR metoprolol, and 0.125-1 mg tesofensine, for example 10-60 mg ER metoprolol, 0.25-15 mg IR metoprolol, and 0.125-0.75 mg tesofensine.
19. The method according to any of the preceding items, comprising administering 10-125 mg ER metoprolol and 0.125-1.5 mg tesofensine; for example 10-100 mg ER metoprolol and 0.125-1 mg tesofensine, for example 12.5-75 mg ER metoprolol and 0.125-0.75 mg tesofensine.
20. The method according to any of the preceding items, wherein tesofensine and the beta blocker are administered separately.
21. The method according to any of the preceding items, wherein tesofensine and the beta blocker are administered in combination.
22. The method according to any of the preceding items, wherein the pharmaceutical composition is administered one, two or three times daily.
23. The method according to any of the preceding items, wherein the subject has a BMI of at least 25 kg/m², such as at least 30, for example at least 35.
24. The method according to any of the preceding items, wherein the subject is diabetic.
25. The method according to item 24, wherein the serum HbA1c level is reduced by at least 10 mmol/mol, such as at least 20, for example at least 25, such as at least 30, for example at least 35 mmol/mol after 24 weeks of treatment.
26. The method according to any of the preceding items, wherein the body weight of the patient is reduced by at least 3% after six months of treatment, such as between 5% and 10% or between 6% and 8%.
27. The method according to any of the preceding items, wherein the waist circumference of the patient is reduced by at least 4 cm after 6 months of treatment, such as between 4 and 6 cm or between 6 and 10 cm.
28. The method according to any of the preceding items, wherein the fat mass of the patient is reduced by at least 2 kg after 6 months of treatment, such as between 2 and 8 kg, or between 3 and 6 kg.
29. The method according to any of the preceding items, wherein the treatment reduces the amount of visceral fat.
30. The method according to any of the preceding items, wherein the treatment reduces one or more symptoms of pre-diabetes, metabolic syndrome, dyslipidemia, atherosclerosis, overeating, bulimia nervosa, binge eating disorder, compulsive over-eating, impaired appetite regulation, nonalcoholic fatty liver disease (NAFLD) and nonalcoholic steatohepatitis (NASH).
31. A method for reducing body weight in a patient suffering from Hypothalamic obesity, the method comprising daily administration of a pharmaceutical composition comprising 0.01 to 1.5 mg tesofensine or a pharmaceutically acceptable salt to said patient.
32. A method for reducing waist circumference in a patient suffering from Hypothalamic obesity, the method comprising daily administration of a pharmaceutical composition comprising 0.01 to 1.5 mg tesofensine or a pharmaceutically acceptable salt to said patient.
33. A method for reducing body fat in a patient suffering from Hypothalamic obesity, the method comprising daily administration of a pharmaceutical composition comprising 0.01 to 1.5 mg tesofensine or a pharmaceutically acceptable salt to said patient.
34. The method of item 33, wherein said body fat is visceral fat.
35. A method for reducing liver fat in a patient suffering from Hypothalamic obesity, the method comprising daily administration of a pharmaceutical composition comprising 0.01 to 1.5 mg tesofensine or a pharmaceutically acceptable salt to said patient.
36. A method for reducing serum HbA1c level in a patient suffering from Hypothalamic obesity, the method comprising daily administration of a pharmaceutical composition comprising 0.01 to 1.5 mg tesofensine or a pharmaceutically acceptable salt to said patient.
37. The method of item 36, wherein the subject suffers from type 2 diabetes, pre-diabetes, metabolic syndrome, insulin resistance, or glucose intolerance, preferably type 2 diabetes.
38. Use of a pharmaceutical composition comprising 0.01 to 1.5 mg tesofensine, or a pharmaceutically acceptable salt thereof, in the manufacture of a medicament for treatment of hypothalamic obesity.

### EXAMPLES

### Example 1. Phase 2 Clinical Trial

The overall safety and tolerability of co-administration of tesofensine and metoprolol (Tesomet) in subjects with hypothalamic injury-induced obesity (HIO) was studied in a phase 2, double-blind, randomized, placebo-controlled, single-center safety and efficacy study followed by with an open-label extension, in total 48 weeks:
Part 1:24 Weeks double blinded treatment followed by
Part 2:24 weeks open lable extension.

### Subjects

The studied population were subjects suffering from obesity developed in relation to damage to the hypothalamus (HO), whether it is from an injuring trauma, bleeding, infaction, tumor, surgery or irradiation.

Modified Intention-to-Treat Population (mITT):

Includes all randomized subjects that have non-missing baseline assessment and at least one post-baseline assessment. Subjects were included into mITT population separately for the double-blind and open-label phase. Subjects in the mITT Population contribute to the evaluation ‘as randomized’.

### Per Protocol Population (PPP):

Includes all randomized subjects without any major protocol violations. Subjects in the PPP contribute to the evaluation ‘as randomized’.

21 subjects were studied, of which 13 subjects received IMP and 8 subjects received placebo treatment. All participants were white, not hispanic or latino. Five participants were male (3 received IMP, 2 received placebo) and 16 participants were female (10 received IMP, 6 received placebo).

TABLE 5
Subjects.

| | | |
|---|---|---|
| Active treatment | Placebo | Total |
| (N = 13) | (N = 8) | (N = 21) |

Age (years)

| | | | |
|---|---|---|---|
| Mean (SD) | 46.9 (12.55) | 44.4 (18.27) | 46.0 (14.59) |
| Median | 51.0 | 43.0 | 50.0 |
| Max, min | 27, 70 | 25, 67 | 25, 70 |

Height (cm)

| | | | |
|---|---|---|---|
| Mean (SD) | 175.45 (7.869) | 171.29 (10.832) | 173.87 (9.084) |
| Median | 175.80 | 169.35 | 175.00 |
| Max, min | 161.5, 188.7 | 158.7, 190.5 | 158.7, 190.5 |

Weight (kg)

| | | | |
|---|---|---|---|
| Mean (SD) | 114.34 (18.828) | 111.64 (27.445) | 113.31 (21.866) |
| Median | 121.10 | 110.00 | 117.40 |
| Max, min | 86.3, 146.8 | 79.7, 164.8 | 79.7, 164.8 |

BMI (kg/m²)

| | | | |
|---|---|---|---|
| Mean (SD) | 37.13 (5.653) | 37.60 (5.836) | 37.31 (5.581) |
| Median | 36.30 | 37.25 | 36.30 |
| Max, min | 27.7, 45.3 | 31.1, 45.5 | 27.7, 45.5 |

### Methodology

This randomized, double-blind, placebo-controlled Phase 2 trial evaluated Tesomet (tesofensine 0.5 mg+metoprolol 50 mg) administered daily in patients with HO, conducted at Rigshospitalet in Copenhagen, Denmark.

The primary endpoint of the study was overall safety and tolerability measured by all safety data collected during the study including recorded adverse events, laboratory data, blood pressure, and heart rate. The efficacy endpoints included bodyweight; body composition; waist circumference, satiety and appetite; lipids and glycemic control; quality of life; and craving for sweet, salty and fatty foods.

### Part 1—Double Blind:

Patients received either Tesomet or matching placebo (2:1 randomization) for 24 weeks.

Active medication arm: co-administration of 0.5 mg tesofensine/50 mg metoprolol ER daily for 24 weeks. One tablet for each product.

Placebo arm: matching placebo tablets daily for 24 weeks

### Part 2—Open Label:

All subjects: co-administration of 0.5 mg tesofensine/50 mg metoprolol ER daily for 24 weeks. One tablet for each product.

The term “IMP” stands for Investigation Medicinal Product and corresponds to the co-administration of tesofensine (0.5 mg)/metoprolol (50 mg).

### Results

18 of the 21 study participants completed the placebo-controlled part of the study (2 dropouts in placebo group; 1 dropout in treatment group).

Continuous efficacy secondary endpoints were compared between treatment arms by means using Analysis of Co-variance (ANCOVA) including treatment as fixed factor and baseline value as covariate. Estimates and 95% confidence intervals of treatment differences were calculated

### Safety

Tesomet was found to be safe and well tolerated. Side effects seen more frequently in treated patients include sleep problems, dry mouth, and headache, which are well known side effects associated with tesofensine and/or metoprolol. There was a single case of Tesomet related anxiety/paranoia reported as a Serious Adverse Event (SAE), which improved after discontinuation of treatment. Notably there was no clinically meaningful difference in heart rate or blood pressure between the treatment groups, proving that the amounts of Tesofensine and Metoprolol were well balanced.

### Body Weight

Treatment with Tesomet led to a statistically significant 6.8% average reduction in bodyweight compared to placebo (p<0.001). The data is presented in Table 6 and FIG. 1.

TABLE 6
Body weight measurements.

| | | | |
|---|---|---|---|
| Weight | Active treatment | Placebo | Total |
| (kg) | (N = 13) | (N = 8) | (N = 21) |

Baseline

| | | | |
|---|---|---|---|
| Mean (SD) | 114.34 (18.828) | 111.64 (27.455) | 113.31 (21.866) |
| Median | 121.10 | 110.00 | 117.40 |
| Max, min | 86.3, 146.8 | 79.7, 164.8 | 79.7, 164.8 |

Visit 8 (24 w)

| | | | |
|---|---|---|---|
| Mean(SD) | 106.76 (19.458) | 111.76 (27.428) | 108.67 (22.286) |
| Median | 111.10 | 110.80 | 110.10 |
| Max, min | 77.2, 132.9 | 77.9, 163.0 | 77.2, 163.0 |

Change from baseline to visit 8

| | | | |
|---|---|---|---|
| Mean (SD) | −7.577 (6.8781) | 0.125 (4.4210) | −4.643 (7.0650) |
| Median | −8.400 | −0.150 | −3.100 |
| Max, min | −20.4, 4.30 | −7.40, 6.20 | −20.4, 6.20 |

### Waist Circumference

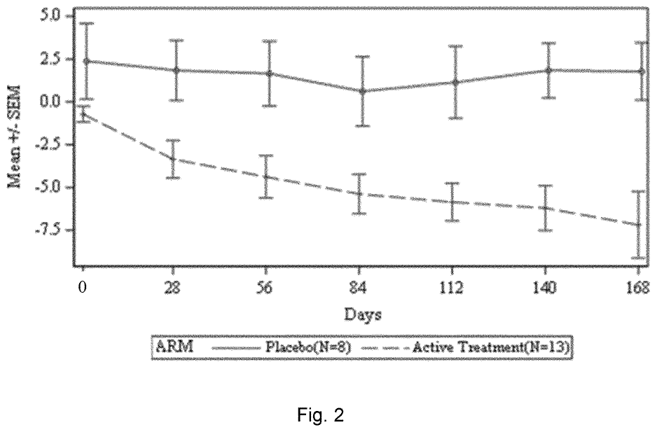

Average waist circumference of Tesomet treated patients was significantly reduced by 7.9% compared to placebo (p<0.001). The data is presented in Table 7 and FIG. 2.

TABLE 7
Waist circumference.

| | | | |
|---|---|---|---|
| Waist | Active | | |
| circumference | treatment | Placebo | Total |
| (cm) | (N = 13) | (N = 8) | (N = 21) |

Baseline

| | | | |
|---|---|---|---|
| Mean (SD) | 118.2 (11.56) | 113.4 (19.16) | 116.4 (14.65) |
| Median | 117.0 | 109.0 | 117.0 |
| Max, min | 102, 142 | 88, 142 | 88, 142 |

Visit 8 (24 w)

| | | | |
|---|---|---|---|
| Mean(SD) | 110.9 (14.83) | 115.3 (17.65) | 112.6 (15.68) |
| Median | 111.0 | 110.5 | 111.0 |
| Max, min | 94, 142 | 94, 142 | 94, 142 |

Change from baseline to visit 8

| | | | |
|---|---|---|---|
| Mean (SD) | −7.308 (7.0165) | 1.875 (4.9982) | −3.810 (7.6917) |
| Median | −8.000 | 2.000 | −3.000 |
| Max, min | −20.0, 5.00 | −5.00, 8.22 | −20.0, 8.00 |

### Body Composition-Fat Mass

Treatment with Tesomet led to a decrease (NS, P=0.0988) in body fat mass as compared to placebo. The data is presented in Table 8.

TABLE 8
Fat mass measurements.

| | | | |
|---|---|---|---|
| Fat mass | Active treatment | Placebo | Total |
| (kg) | (N = 13) | (N = 8) | (N = 21) |

Baseline

| | | | |
|---|---|---|---|
| Mean (SD) | 51.665 (15.0417) | 48.560 (13.2804) | 50.482 (14.1375) |
| Median | 51.370 | 47.300 | 51.370 |
| Max, min | 31.46, 70.38 | 32.75, 67.56 | 31.46, 70.38 |

End of part 1

| | | | |
|---|---|---|---|
| Mean(SD) | 45.758 (14.3925) | 49.042 (13.9910) | 46.853 (13.9335) |
| Median | 45.905 | 48.405 | 45.905 |
| Max, min | 28.91, 71.50 | 32.50, 65.43 | 28.91, 71.50 |

Change from baseline to end of part 1

| | | | |
|---|---|---|---|
| Mean (SD) | −5.307 (5.3426) | −1.045 (3.7498) | −3.887 (5.1846) |
| Median | −4.395 | −1.670 | −1.950 |
| Max, min | −15.3, 2.40 | −6.75, 4.29 | −15.3, 4.29 |

### Glycaemic Control (HbA1c)

Tesomet treatment improved glycemic control as measured by a statistically significant 14.6% reduction in hemoglobin A1c (HbA1c) compared to placebo (p=0.015). The data is presented in Table 9.

TABLE 9
Glycaemic Control (HbA1c) measurements.

| | | | |
|---|---|---|---|
| HbA1c | Active treatment | Placebo | Total |
| (mmol/mol) | (N = 13) | (N = 8) | (N = 21) |

Baseline

| | | | |
|---|---|---|---|
| n | 12 | 7 | 19 |
| Mean (SD) | 41.583 (17.2176) | 38.429 (3.2587) | 40.421 (13.6801) |
| Median | 35.500 | 39.000 | 37.000 |
| Max, min | 29.00, 85.00 | 33.00, 42.00 | 29.00, 85.00 |

End of part 1

| | | | |
|---|---|---|---|
| n | 12 | 6 | 18 |
| Mean(SD) | 35.583 (3.5280) | 38.167 (3.6560 | 36.444 (3.6818) |

Change from baseline to end of part 1

| | | | |
|---|---|---|---|
| n | 12 | 6 | 18 |
| Mean (SD) | −6.000 (15.3859) | −0.167 (2.4014) | −4.056 (12.7624) |

### Example 2. Open-Label Extension of Phase 2 Study

TM005 was a 24-week phase 2, double-blind, randomized, placebo-controlled, single-centre, safety and efficacy study followed by a 24-week open-label extension treatment period designed to evaluate overall safety and tolerability of Tesomet (co-administration of 0.5 mg tesofensine and 50 mg metoprolol) in patients with hypothalamic obesity (HO).

The primary endpoint of the study was overall safety and tolerability measured by all safety data collected during the study including recorded adverse events, laboratory data, blood pressure, and heart rate. The secondary efficacy endpoints included bodyweight, waist circumference, glycemic control and other measures. In the double-blind (DB) period of the study, patients received either Tesomet or matching placebo (2:1 randomization) for 24 weeks.

A total of 21 patients (13 Tesomet, 8 placebo) were randomized into the double blind (DB) period. All patients who completed the DB period of the study were provided the opportunity to receive Tesomet in an open-label extension (OLE) period of the study for an additional 24 weeks. All 18 patients who completed the DB period chose to participate in the OLE period and all of these patients completed the OLE period. Patients entering the OLE period were 83.3% female and on average 44.9 years old, weighing 110.4 kg (243 lbs) with a BMI of 37.2 kg/m².

For the open-label extension part of the study, the Tesomet group is still labelled Tesomet in the results. The Placebo group is also labelled Placebo although in the open label extension they also receive Tesomet.

Tesomet was well-tolerated in hypothalamic obesity patients throughout the duration of the 48-week trial, with no clinically meaningful differences in heart rate or blood pressure observed.

Treatment with Tesomet compared to placebo resulted in a statistically significant difference in the number of responders with a ≥5% body weight reduction from baseline to 24 weeks during the double-blind treatment period and this effect was maintained following an additional 24 weeks of open-label Tesomet treatment (FIG. 3a). Patients treated with placebo during the first 24 week double-blind period of the study and that then received 24 weeks of Tesomet treatment during the open-label extension period also showed a marked improvement in the number of responders with ≥5% body weight reduction from baseline.

Treatment with Tesomet compared to placebo resulted in a significant difference in the number of responders with a ≥10% body weight reduction from baseline to 24 weeks during the double-blind treatment period and this effect remained high following an additional 24 weeks of open-label Tesomet treatment (FIG. 3b). Patients treated with placebo during the first 24 week double-blind period of the study and that then received 24 weeks of Tesomet treatment during the open-label extension period also showed a marked improvement in the number of responders with ≥10% body weight reduction from baseline.

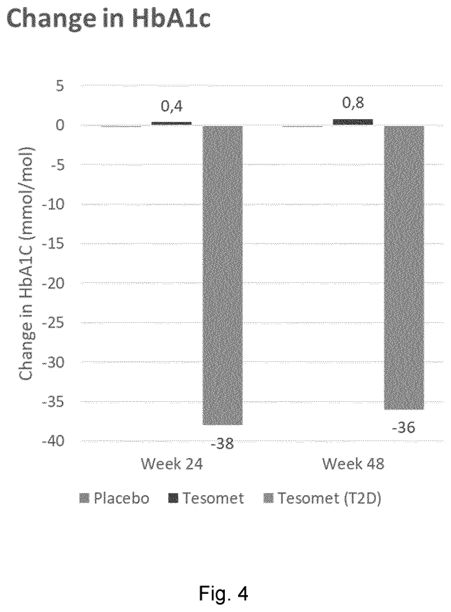

Tesomet treatment resulted in a clinically meaningful reductions in HbA1c levels in patients with Type-2 diabetes after 24 and 48 weeks of treatment, whereas no effect was seen on normoglycemic patients (FIG. 4).

Patients receiving Tesomet for the full 48 weeks of the study demonstrated statistically significant and clinically meaningful reductions in body weight and waist circumference from baseline to Week 48, as well as improvements in glycemic control. Improvements observed in the DB period of the study were maintained over the duration of the OLE period (FIG. 5).

Patients who received placebo in the DB period of the study and were subsequently switched to Tesomet for the OLE period also achieved clinically meaningful reductions in body weight, waist circumference (data not shown) and BMI (data not shown) after being switched to Tesomet during the 24-week OLE period (see FIG. 5).

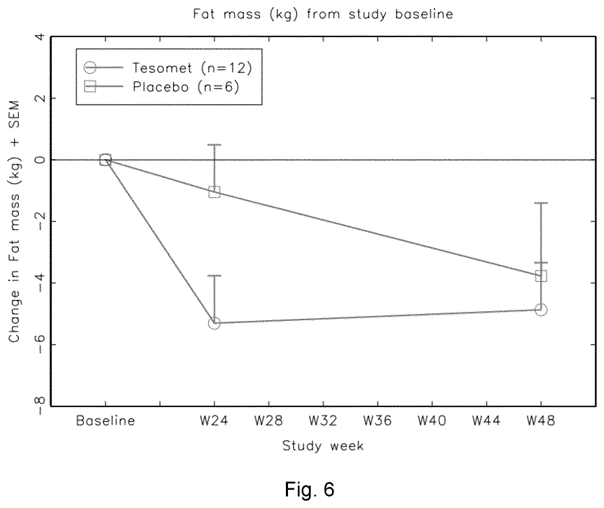

Treatment with Tesomet compared to placebo resulted in a clinically meaningful reduction in fat mass from baseline to 24 weeks of treatment and this effect was maintained following an additional 24 weeks of open-label Tesomet treatment. Patients treated with placebo during the first 24 weeks of the study and that then received 24 weeks of open-label Tesomet treatment also showed clinically meaningful reductions in fat mass from baseline (FIG. 6).

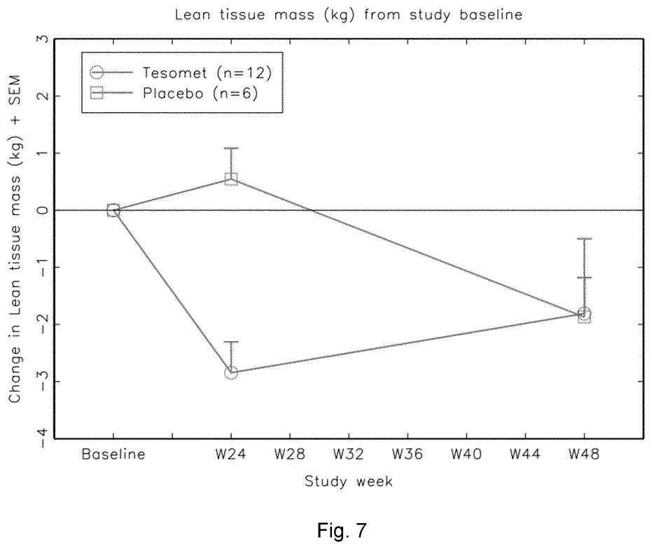

Patients treated with Tesomet during the 24 week double-blind phase followed by an additional 24 weeks of open-label Tesomet treatment showed evidence of increased lean tissue mass during the open-label extension (FIG. 7). In contrast, patients receiving placebo during the 24 week double-blind phase followed by 24 weeks of open-label Tesomet treatment showed evidence of decreased lean tissue mass. The same tendency for lean mass loss was seen for Tesomet patients during the 24 weeks of the double-blind phase.

In summary, the results of the OLE period of the study reinforce the striking and positive effect of Tesomet on body weight, body composition, and metabolic dysregulation observed in the DB period of the Phase 2 study in patients with hypothalamic obesity.

1-35. (canceled)

36. A method for treating hypothalamic obesity in a subject in need thereof comprising a treatment comprising administering to the subject one time daily a pharmaceutical composition comprising:
0.01 to 1.5 mg tesofensine, or a pharmaceutically acceptable salt thereof;
wherein the subject has a body mass index (BMI) of greater than 30 kg/m² prior to the treatment; and
wherein the fat mass of the subject is reduced by at least 2 kg after 6 months of the treatment.

37. (canceled)

38. (canceled)

39. The method according to claim 36, wherein the subject has a body mass index (BMI) of at least 35 kg/m² prior to the treatment.

40. (canceled)

41. The method according to claim 36, wherein the serum HbA1c of the subject is reduced by at least 10 mmol/mol after 24 weeks of the treatment.

42. The method according to claim 36, wherein the body weight of the subject is reduced by at least 3% after six months of the treatment.

43. The method according to claim 36, wherein the waist circumference of the subject is reduced by at least 4 cm after six months of the treatment.

44. The method according to claim 36, wherein the fat mass includes visceral fat.

45. The method according to claim 36, wherein the tesofensine or pharmaceutically acceptable salt thereof is the free base, the citrate salt, or the tartrate salt.

46. The method according to claim 36, wherein the pharmaceutical composition is administered daily.

47. (canceled)

48. (canceled)

49. (canceled)

50. The method according to claim 36, wherein the daily dose of tesofensine or a pharmaceutically acceptable salt thereof is less than 1 mg of tesofensine.

51. A method for reducing the serum HbA1c level in a subject suffering from hypothalamic obesity, wherein said method comprises administering to the subject one time daily a pharmaceutical composition comprising 0.01 to 1.5 mg of tesofensine or a pharmaceutically acceptable salt thereof; wherein the subject has a body mass index (BMI) of at least 30 kg/m² prior to the administration of the pharmaceutical composition.

52. The method according to claim 51, wherein the serum Hb1Ac level is reduced by at least 10 mmol/mol after 24 weeks of treatment.

53. A method for reducing the body weight in a subject suffering from hypothalamic obesity, wherein said method comprises a treatment comprising administering to the subject one time a day a pharmaceutical composition comprising 0.01 to 1.5 mg of tesofensine or a pharmaceutically acceptable salt thereof; wherein the subject has a body mass index (BMI) of at least 30 kg/m² prior to the treatment;.

54. The method according to claim 53, wherein the body weight of the subject is reduced by at least 3% after six months of treatment.

55. The method according to claim 53, wherein the fat mass of the subject is reduced by at least 2 kg after six months of treatment.

56. The method according to claim 36, wherein the subject is diabetic prior to the treatment.

57. The method according to claim 36, wherein the subject has a waist circumference of at least 102 cm prior to the treatment.

58. The method according to claim 36, wherein the subject has a body mass index (BMI) of at least 35 kg/m² prior to the treatment.

59. The method according to claim 36, wherein the subject has a fat mass of at least 3146 kg prior to the treatment.

60. The method according to claim 36, wherein the subject has an HbA1c of at least 29 mmol/mol prior to the treatment.
